SEQUENCE LISTING (63 sequences)

SEQ ID NO: 1 - protein, 10 aa, synthetic construct
  REGION 1..10: note = CDRH1
GYSFTSYWIG

SEQ ID NO: 2 - protein, 10 aa, synthetic construct
  REGION 1..10: note = CDRH2
VIYPGDSYTR

SEQ ID NO: 3 - protein, 9 aa, synthetic construct
  REGION 1..9: note = CDRH3
  SITE 1: note = MISC_FEATURE - Xaa denotes Phe, Met, Gln, Leu or Val
  SITE 6: note = MISC_FEATURE - Xaa denotes Ser or Ala
  SITE 7: note = MISC_FEATURE - Xaa denotes Phe, Leu, Ala or Met
  SITE 9: note = MISC_FEATURE - Xaa denotes Tyr, Gln, Lys, Arg, Trp, His, Ala, Thr, Ser, Asn orGly
XPNWGXXDX

SEQ ID NO: 4 - protein, 9 aa, synthetic construct
  REGION 1..9: note = CDR H3 SEQUENCE
FPNWGALDQ

SEQ ID NO: 5 - protein, 9 aa, synthetic construct
  REGION 1..9: note = CDR H3 SEQUENCE
VPNMGSLDT

SEQ ID NO: 6 - protein, 9 aa, synthetic construct
  REGION 1..9: note = CDR H3 SEQUENCE
FPNWGSMDA

SEQ ID NO: 7 - protein, 9 aa, synthetic construct
  REGION 1..9: note = CDR H3 SEQUENCE
FPNWGSLDH

SEQ ID NO: 8 - protein, 9 aa, synthetic construct
  REGION 1..9: note = CDR H3 SEQUENCE
MPNWGSFDY

SEQ ID NO: 9 - protein, 9 aa, synthetic construct
  REGION 1..9: note = CDR H3 SEQUENCE
MPNWGSFDT

SEQ ID NO: 10 - protein, 9 aa, synthetic construct
  REGION 1..9: note = CDR H3 SEQUENCE
MPNWGSLDH

SEQ ID NO: 11 - protein, 9 aa, synthetic construct
  REGION 1..9: note = CDR H3 SEQUENCE
MPNWGSFDS

SEQ ID NO: 12 - protein, 9 aa, synthetic construct
  REGION 1..9: note = CDR H3 SEQUENCE
MPNWGSLDT

SEQ ID NO: 13 - protein, 9 aa, synthetic construct
  REGION 1..9: note = CDR H3 SEQUENCE
MPNWGSLDA

SEQ ID NO: 14 - protein, 9 aa, synthetic construct
  REGION 1..9: note = CDR H3 SEQUENCE
MPNWGSLDN

SEQ ID NO: 15 - protein, 9 aa, synthetic construct
  REGION 1..9: note = CDR H3 SEQUENCE
MPNWGALDS

SEQ ID NO: 16 - protein, 9 aa, synthetic construct
  REGION 1..9: note = CDR H3 SEQUENCE
MPNWGSLDN

SEQ ID NO: 17 - protein, 9 aa, synthetic construct
  REGION 1..9: note = CDR H3 SEQUENCE
MPNWGSLDY

SEQ ID NO: 18 - protein, 9 aa, synthetic construct
  REGION 1..9: note = CDR H3 SEQUENCE
MPNWGSFDH

SEQ ID NO: 19 - protein, 9 aa, synthetic construct
  REGION 1..9: note = CDR H3 SEQUENCE
MPNWGSLDS

SEQ ID NO: 20 - protein, 9 aa, synthetic construct
  REGION 1..9: note = CDR H3 SEQUENCE
MPNWGSLDG

SEQ ID NO: 21 - protein, 9 aa, synthetic construct
  REGION 1..9: note = CDR H3 SEQUENCE
VPNWGSLDG

SEQ ID NO: 22 - protein, 28 aa, synthetic construct
  REGION 1..28: note = CDR H3 SEQUENCE
CARFPNWGSLDHWGQGTLVTVSSASIKG

SEQ ID NO: 23 - protein, 28 aa, synthetic construct
  REGION 1..28: note = CDR H3 SEQUENCE
CARMPNWGSLDHWGQGTLVTVSSASTKG

SEQ ID NO: 24 - protein, 28 aa, synthetic construct
  REGION 1..28: note = CDR H3 SEQUENCE
CARMPNWGSFDYWGQGTLVTVSSASIKG

SEQ ID NO: 25 - protein, 12 aa, synthetic construct
  REGION 1..12: note = CDR H3 SEQUENCE
VRMPNWGSLDHW

SEQ ID NO: 26 - protein, 27 aa, synthetic construct
  REGION 1..27: note = CDR H3 SEQUENCE
VRMPNWGSLDHWGQGTLVTVSSADIKG

SEQ ID NO: 27 - protein, 27 aa, synthetic construct
  REGION 1..27: note = CDR H3 SEQUENCE
ARMPNWGSLDHWGQGTLVTVSSASIKG

SEQ ID NO: 28 - protein, 25 aa, synthetic construct
  REGION 1..25: note = CDR H3 SEQUENCE
FPNWGSFDYWGQGTLVTVSSASIKG

SEQ ID NO: 29 - protein, 9 aa, synthetic construct
  REGION 1..9: note = CDR H3 SEQUENCE
VPNWGSLDA

SEQ ID NO: 30 - protein, 9 aa, synthetic construct
  REGION 1..9: note = CDR H3 SEQUENCE
FPNWGSFDY

SEQ ID NO: 31 - protein, 12 aa, synthetic construct
  REGION 1..12: note = CDRL1
RASQSISSSYLA

SEQ ID NO: 32 - protein, 7 aa, synthetic construct
  REGION 1..7: note = CDRL2
GASSRAT

SEQ ID NO: 33 - skipped

SEQ ID NO: 34 - protein, 5 aa, synthetic construct
  REGION 1..5: note = CDRL3 SEQUENCE
QRYAT

SEQ ID NO: 35 - protein, 5 aa, synthetic construct
  REGION 1..5: note = CDRL3 SEQUENCE
QRYST

SEQ ID NO: 36 - protein, 5 aa, synthetic construct
  REGION 1..5: note = CDRL3 SEQUENCE
QMYST

SEQ ID NO: 37 - protein, 5 aa, synthetic construct
  REGION 1..5: note = CDRL3 SEQUENCE
QQVGT

SEQ ID NO: 38 - protein, 5 aa, synthetic construct
  REGION 1..5: note = CDRL3 SEQUENCE
QQVST

SEQ ID NO: 39 - protein, 5 aa, synthetic construct
  REGION 1..5: note = CDRL3 SEQUENCE
QQYST

SEQ ID NO: 40 - protein, 5 aa, synthetic construct
  REGION 1..5: note = CDRL3 SEQUENCE
QSYST

SEQ ID NO: 41 - protein, 5 aa, synthetic construct
  REGION 1..5: note = CDRL3 SEQUENCE
QQYAT

SEQ ID NO: 42 - protein, 5 aa, synthetic construct
  REGION 1..5: note = CDRL3 SEQUENCE
QQYSS

SEQ ID NO: 43 - protein, 5 aa, synthetic construct
  REGION 1..5: note = CDRL3 SEQUENCE
QTYST

SEQ ID NO: 44 - protein, 5 aa, synthetic construct
  REGION 1..5: note = CDRL3 SEQUENCE
QQYGS

SEQ ID NO: 45 - protein, 5 aa, synthetic construct
  REGION 1..5: note = CDRL3 SEQUENCE
QQYAS

SEQ ID NO: 46 - protein, 5 aa, synthetic construct
  REGION 1..5: note = CDRL3 SEQUENCE
QQYEA

SEQ ID NO: 47 - protein, 5 aa, synthetic construct
  REGION 1..5: note = CDRL3 SEQUENCE
QQYET

SEQ ID NO: 48 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 49 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 50 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 51 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 52 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 53 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 54 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 55 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 443 aa, synthetic construct
  REGION 1..443: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 61 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0078192 A1
(43) Pub. Date: Mar. 19, 2026

(54) TREATMENT FOR SLEEP LOSS OR SLEEP DISTURBANCE IN PATIENTS WITH DERMATITIS

(71) Applicant: ASLAN PHARMACEUTICALS PTE LTD, Singapore (SG)

(72) Inventor: Karen VEVERKA, San Mateo, CA (US)

(21) Appl. No.: 19/109,353

(22) PCT Filed: Sep. 6, 2023
(86) PCT No.: PCT/SG2023/050606

§ 371 Date: Mar. 6, 2025

Related U.S. Application Data
(60) Provisional application No. 63/374,678, filed on Sep. 6, 2022.

(51) Int. Cl. C07K 16/28; A61P 17/04

(52) U.S. Cl. CPC C07K 16/2866; A61P 17/04

(57) ABSTRACT

An anti-IL-13Rα1 antibody or an antigen binding fragment thereof and pharmaceutical formulations comprising same for use in treating patients with skin disorders, like atopic dermatitis, experiencing sleep loss or sleep disturbance caused by pruritus. Also provided is a method of treating sleep loss or sleep disturbance caused by pruritus in said patients, comprising administering an anti-IL-13Rα1 antibody or an antigen binding fragment thereof or a pharmaceutical formulation comprising the same to a patient in need thereof.

The present disclosure relates to use of an anti-IL-13Rα1 antibody or a binding fragment thereof and pharmaceutical formulations comprising same to treat patients experiencing sleep loss or sleep disturbance.

### BACKGROUND

It is common for all individuals to occasionally experience bouts of sleep loss or sleep disturbance, for example due to stress and anxiety, shift work which may disrupt sleep cycles, the use of certain medications, etc. However, a subset of individuals, such as highly allergic patients, regularly experience sleep loss or sleep disturbances due to their underlying medical conditions.

Highly allergic patients often have skin disorders, such as atopic dermatitis or eczema. Chronic itch is a hallmark and major symptom of atopic dermatitis and other type 2-driven inflammatory skin disorders. These conditions which therefore may make it particularly difficult for these patients to fall asleep or stay asleep due to the discomfort they experience on a daily basis.

Itch signalling in atopic dermatitis (AD) has been recently postulated to be exacerbated by pro-inflammatory cytokines present in the skin, causing an immune response that disrupts the skin barrier and drives disease pathology. For some cytokines such as IL-31 and IL-4, their role in itch signalling has been well documented. However, for other cytokines, such as IL-13, there is less evidence of a direct role in the pathophysiology of pruritus.

One way to inhibit the activity of IL-13 is to interfere with the binding of IL-13 to its receptor IL-13R, for example by using an antibody specific to IL-13R, such as an antibody specific to IL-13Rα1. An effective antibody antagonist to IL-13Rα1 may also interfere with the binding of IL-13 and prevent heterodimerization of IL-4Rα and IL-13Rα1. Such an antibody could inhibit signaling of both IL-13 and IL-4 through the type II receptor while sparing IL-4 signalling through the type I receptor. Signalling through the type I receptor is essential in the induction phase of the immune response during which Th2 cells differentiate. T cells do not express IL-13Rα1 so the type II receptor plays no role in Th2 differentiation. Hence, an IL-13Rα1 antibody should not affect the overall Th1/Th2 balance. Signalling through the type II IL-4/IL-13 receptor is critical during the effector-A-stage of the immune response during established allergic inflammation. Thus, blockade of the type II receptor should have a beneficial effect on many of the symptoms of conditions mediated by IL-13R-mediated and therefore, be an effective disease modifying agent.

Antibodies against IL-13Rα1 (both monoclonal and polyclonal) have been described in the art; see, eg, WO 97/15663, WO 03/80675; WO 03/46009; WO 06/072564; Gauchat et al, 1998 Eur. J. Immunol. 28:4286-4298; Gauchat et al, 2000 Eur. J. Immunol. 30:3157-3164; Clement et al, 1997 Cytokine 9(11):959 (Meeting Abstract); Ogata et al, 1998 J. Biol. Chem. 273:9864-9871; Graber et al, 1998 Eur. J. Immunol. 28:4286-4298; C. Vermot-Desroches et al, 2000 Tissue Antigens 5(Supp. 1):52-53 (Meeting Abstract); Poudrier et al, 2000 Eur. J. Immunol. 30:3157-3164; Akaiwa et al, 2001 Cytokine 13:75-84; Cancino-Diaz et al, 2002 J. Invest Dermatol. 119:1114-1120; and Krause et al, 2006 MoI. Immunol. 43:1799-1807.

One particularly promising anti-IL-13Rα1 antibody is described in WO2008/060813 as antibody 10G5-6. 10G5-6 is an IgG4 with a hinge stabilising serine to proline mutation (S241P Kabat numbering). This antibody is now known as Eblasakimab (previously called ASLAN004, both names used interchangeably herein). Eblasakimab has been shown to bind to human IL-13Rα1 with a high affinity (for example Kd may be 500pM). Eblasakimab was shown to effectively antagonise IL-13 function through inhibiting the binding of IL-13 to its receptor IL-13Rα1 and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells.

Long term sleep loss is known to result in the impairment of cognitive abilities, increased irritability, sleepiness during the day, all of which have a significant negative impact on quality of life. Furthermore, the cumulative long-term effects of sleep loss and sleep disorders have also been associated with a wide range of deleterious health consequences such as an increased risk of diabetes, depression, heart attack, and stroke.

Accordingly, there is a need for alternative treatments in order to manage and treat sleep loss or sleep disturbance, for example sleep loss or sleep disturbance in highly allergic patients.

### SUMMARY OF THE DISCLOSURE

The following paragraphs summarise the present disclosure:
1. An antibody or antigen binding fragment thereof, which is an inhibitor of signalling through IL-13Rα1 by binding the said receptor, for use in the treatment of sleep loss or sleep disturbance in a patient by parenteral administration of a treatment cycle comprising a dose in the range 200 mg to 600 mg, (for example 300, 400, 500 or 600 mg, such as 400 to 600 mg), for example wherein the treatment results in a reduction in the sleep disturbance (SD) score of the Patient Oriented Eczema Measure (POEM), for example in the range −50 to −100% from the baseline, such as a reduction of 2 to 4 points from the baseline.
1A. An antibody or antigen binding fragment thereof, which is an inhibitor of signalling through IL-13Rα1 by binding the said receptor, for use in the reduction of the sleep disturbance (SD) score of the Patient Oriented Eczema Measure (POEM), for example to reduce the sleep disturbance score in the range −50 to −100%, such as a reduction of 2 to 4 points from the baseline, in a patient with sleep loss by parenteral administration of a treatment cycle comprising a dose in the range 200 mg to 600 mg, (such as 400 to 600 mg).
1B. An antibody or antigen binding fragment thereof, which is an inhibitor of signalling through IL-13Rα1 by binding the said receptor, for use in the reduction of the sleep deprivation component of SCORAD, for example in the range of −20 to −100% from the baseline, such as a reduction of 2 or more points, such as 2, 3, 4, 5, 6, 7, 8, 9 or 10 points from the baseline.
1C. An antibody or antigen binding fragment thereof, which is an inhibitor of signalling through IL-13Rα1 by binding the said receptor, for use in the reduction of the sleep disturbance numerical rating scale (SD NRS), ), for example in the range of −20 to −100% from the baseline, such as a reduction of 4 or more points, such as 4, 5, 6, 7, 8, 9 or 10 points from the baseline.
2. An antibody or antigen binding fragment thereof according to any one of the preceding paragraphs, wherein the treatment results in a reduction in the sleep disturbance (SD) score of the Patient Oriented Eczema Measure (POEM), for example in the range −50 to −100% from the baseline, such as a reduction of 2 to 4 points from the baseline.
3. An antibody or antigen binding fragment thereof for use according to any one of the preceding paragraphs, wherein the patient has a baseline SD score of at least 3, such as 3 or 4.
4. An antibody or antigen binding fragment thereof for use according to any one of the preceding paragraphs, wherein the patient has a baseline SD score of 3.
5. An antibody or antigen binding fragment thereof for use according to any one of the preceding paragraphs, wherein the patient has a baseline SD score of 4.
6. An antibody or antigen binding fragment thereof for use according any one of the preceding paragraphs, wherein the reduction in SD score is 2 to 4 points from the baseline, such as 2, 3 or 4 point reduction.
7. An antibody or antigen binding fragment thereof for use according any one of the preceding paragraphs, wherein the reduction in SD score is 2 points from the baseline.
8. An antibody or antigen binding fragment thereof for use according any one of the preceding paragraphs, wherein the reduction in SD score is 3 points from the baseline.
9. An antibody or antigen binding fragment thereof for use according any one of the preceding paragraphs, wherein the reduction in SD score is 4 points from the baseline.
10. An antibody or antigen binding fragment thereof for use according to any one of the preceding paragraphs, wherein the reduction in SD score is at least −50% from the baseline, for example a reduction of 2 points from a baseline SD score of 4.
11. An antibody or antigen binding fragment thereof for use according to any one of the preceding paragraphs, wherein the reduction in SD score is at least −66% from the baseline, for example a reduction of 2 points from a baseline SD score of 3.
12. An antibody or antigen binding fragment thereof for use according to any one of the preceding paragraphs, wherein the reduction in SD score is at least −75% from the baseline, for example a reduction of 3 points from a baseline SD score of 4.
13. An antibody or antigen binding fragment thereof for use according to any one of the preceding paragraphs, wherein the reduction in SD score is 100% from the baseline, for example a reduction of 2 points from a baseline SD score of 2, a reduction of 3 points from a baseline SD score of 3, or a reduction of 4 points from a baseline SD score of 4.
14. An antibody or antigen binding fragment thereof for use according to any one of paragraphs 1 to 13, wherein the treatment results in a reduction in the sleep deprivation component of SCORAD, for example a reduction of −20% to −100% from the baseline, such as a reduction of 2 to 10 points from the baseline.
15. An antibody or antigen binding fragment therefor for use according to paragraph 14, wherein the treatment results in a reduction in at least −30% from the baseline in the sleep deprivation component of SCORAD, such as −30%, −35%, −40%, −45%, −50%, −55%, −60%, −65%, −70%, −75%, −80%, −85%, −90%, −95% or −100%.
16. An antibody or antigen binding fragment thereof for use according to paragraphs 14 or 15, wherein the treatment results in a reduction of −35% to −55% from the baseline in the sleep deprivation component of SCORAD, such as −35%, −40%, −50%, or −55%.
17. An antibody or antigen binding fragment therefor for use according to any one of paragraphs 14 to 16, wherein the treatment results in a reduction in the sleep deprivation component of SCORAD of 4 or more points from the baseline, such as 4, 5, 6, 7, 8, 9 or 10 points.
18. An antibody or antigen binding fragment thereof for use according to any one of paragraphs 1 to 17, wherein the patient has a baseline sleep deprivation component of SCORAD of at least 4 points, such as 4, 5, 6, 7, 8, 9 or 10 points.
19. An antibody or antigen binding fragment thereof for use according to any one of paragraphs 1 to 18, wherein the treatment results in a reduction in the sleep disturbance numerical rating scale (SD NRS), for example a reduction of −20% to −100% from the baseline, such as a reduction of 2 to 10 points from the baseline.
20. An antibody or antigen binding fragment therefor for use according to paragraph 19, wherein the treatment results in a reduction in at least −30% from the baseline in SD NRS, such as −30%, −35%, −40%, −45%, −50%, −55%, −60%, −65%, −70%, −75%, −80%, −85%, −90%, −95% or −100%.
21. An antibody or antigen binding fragment therefor for use according to any one of paragraphs 19 to 20, wherein the treatment results in a reduction in SD NRS of 4 or more points from the baseline, such as 4, 5, 6, 7, 8, 9 or 10 points.
22. An antibody or antigen binding fragment thereof for use according to any one of paragraphs 1 to 21, wherein the patient has a baseline sleep disturbance numerical rating scale (SD NRS) of at least 4 points, such as 4, 5, 6, 7, 8, 9 or 10 points.
23. An antibody or antigen binding fragment thereof for use according to any one of the preceding paragraphs, wherein the patient is a highly allergic patient, for example where the baseline baseline IgE levels have been established and are at a level of at least 10,000 KU/L+/−2,000.
24. An antibody or antigen binding fragment thereof for use according to paragraph 23 wherein the baseline IgE levels are in the range 10,000+/−2,000 to 30,000+/−6,000 KU/L.
25. An antibody or antigen binding fragment thereof for use according to any one of paragraphs 23 to 24, wherein the treatment reduces the IgE levels by at least 15% from baseline.
26. An antibody or binding fragment thereof for use according to paragraph 25, wherein the treatment reduces the IgE levels by at least 20% from the baseline.
27. An antibody or binding fragment thereof for use according to paragraph 25 or 26, wherein the treatment reduces the IgE levels by at least 30% from baseline, for example reduces said levels 30 to 40%, such as 30, 31, 32, 33, 34, 35, 36, 37, 38, 39 or 40% from baseline.
28. An antibody or binding fragment according to any one of paragraphs 1 to 27, wherein the reduction is observed by about day 15.
29. An antibody or binding fragment thereof according to any one of paragraphs 1 to 27, wherein the reduction is observed by about day 29.
30. An antibody or binding fragment thereof according to any one of paragraphs 1 to 27, wherein the reduction is observed by about day 57.
31. An antibody or antigen binding fragment thereof for use according to any one of the preceding paragraphs, wherein the patient has pruritus.
32. An antibody or antigen binding fragment thereof for use according to paragraph 21, wherein the pruritus is mediated by one or more of the following: histamine, 5-HT (serotonin), acetylcholine, substance P (SP), leukotrienes, bradykinin, proteases (such as typsin, tryptase, cathepsin S, or kallikrein-related peptidases such as KLK4 or KLK14), IL-31, lysophosphatidic acid, autotaxin and/or toll-like receptor 7 (TLR7).
33. An antibody or antigen binding fragment thereof for use according to any one of paragraphs 31 to 32, wherein the pruritus is mediated by histamine.
34. An antibody or antigen binding fragment thereof for use according to any one of paragraphs 31 to 33, wherein the pruritus is mediated by IL-13, IL-4 or a combination of both.
35. An antibody or antigen binding fragment thereof for use according to any one of paragraphs 31 to 34, wherein the pruritus is mediated by both IL-13 and IL-4.
36. An antibody or antigen binding fragment thereof for use according to any one of the preceding paragraphs, wherein the patient has a skin condition, for example selected from the group comprising dermatitis; such as atopic dermatitis (AD), contact dermatitis, neurodermatitis or seborrheic dermatitis; eczema; hand-foot-and-mouth disease; hives (including urticaria associated with Lupus), psoriasis, an infection, such as a fungal or bacterial infection, for example impetigo or folliculitis; an allergic skin reaction; Ehlers-Danlos syndrome; asthma; and angioedema, such as hereditary angioedema (HAE).
37. An antibody or antigen binding fragment thereof for use according to any one of the preceding paragraphs, wherein the patient has dermatitis, for example atopic dermatitis (AD), contact dermatitis, neurodermatitis or seborrheic dermatitis.
38. An antibody or antigen binding fragment thereof for use according to any one of the preceding paragraphs, wherein the patient has atopic dermatitis (AD), for example moderate to severe atopic dermatitis.
39. An antibody or antigen binding fragment thereof for use according to any one of the preceding paragraphs, wherein the patient has Ehlers-Danlos syndrome (EDS), asthma or angioedema (such as HAE).
40. An antibody or antigen binding fragment thereof for use according to any one of the preceding paragraphs, wherein the patient has asthma.
41. An antibody or antigen binding fragment thereof for use according to any one of the preceding paragraphs, wherein the patient has angioedema.
42. An antibody or antigen binding fragment thereof for use according to any one of paragraphs 1 to 41, wherein the patient does not have atopic dermatitis or psoriasis.
43. An antibody or antigen binding fragment thereof for use according to any one of paragraphs 1 to 42, wherein the patient does not have atopic dermatitis.
44. An antibody or antigen binding fragment thereof for use according to any one of paragraphs 1 to 42, wherein the patient does not have psoriasis.
45. An antibody or antigen binding fragment thereof for use according to any one of paragraphs 1 to 44, wherein the patient was previously administered dupilumab.
46. An antibody or antigen binding fragment thereof for use according to any one of paragraphs 1 to 45, wherein the patient had sleep loss or sleep disturbance that was poorly controlled by dupilumab.
47. An antibody or antigen binding fragment thereof for use according to any one of paragraphs 1 to 46, wherein the patient has atopic dermatitis and had sleep loss or sleep disturbance that was poorly controlled by dupilumab.
48. An antibody or antigen binding fragment thereof for use according to any one of the preceding paragraphs, wherein a reduction in SD score, sleep deprivation component of SCORAD, or SD NRS is present after about two weeks from administration of the first dose (such as day 15).
49. An antibody or antigen binding fragment thereof for use according to any one of paragraphs 1 to 47, wherein a reduction in SD score, sleep deprivation component of SCORAD, or SD NRS is present after about four weeks from administration of the first dose (such as day 29).
50. An antibody or antigen binding fragment thereof for use according to any one of paragraphs 1 to 57, wherein a reduction in SD score, sleep deprivation component of SCORAD, or SD NRS is present after about six weeks from administration of the first dose (such as day 43).
51. An antibody or antigen binding fragment thereof for use according to any one of paragraphs 1 to 57, wherein a reduction in SD score, sleep deprivation component of SCORAD, or SD NRS is present after about eight weeks from administration of the first dose (such as day 57).
52. An antibody or antigen binding fragment thereof for use according to any one of the preceding paragraphs, wherein the treatment is administered intravenously.
53. An antibody or antigen binding fragment thereof for use according to any one of paragraphs 1 to 51, wherein the treatment is administered subcutaneously.
54. An antibody or antigen binding fragment thereof for use according to any one of the preceding paragraphs, wherein multiple doses are administered in a treatment cycle (for example wherein the treatment cycle is 4 to 8 weeks, such as 8 weeks).
55. An antibody or antigen binding fragment thereof for use according to any one of the preceding paragraphs, wherein multiple treatment cycles are administered, for example 2, 3, 4 or more treatment cycles are administered.
56. An antibody or antigen binding fragment thereof for use according to paragraphs 41 or 42 wherein following the treatment cycle or cycles and disease modification, maintenance therapy is administered, for example the same dose administered less frequently (for example monthly), or a lower dose (such as 200 mg) administered the same frequency or less frequently (such as about two weekly, about three weekly, or about four weekly.
57. An antibody or antigen binding fragment thereof for use according to any one of the preceding paragraphs, wherein said antibody or binding fragment thereof is administered approximately weekly, (in particular a single treatment cycle, especially 8 weeks).
58. An antibody or antigen binding fragment thereof for use according to any one of paragraphs 1 to 55, wherein said antibody or binding fragment thereof is administered once approximately every two weeks, (in particular a single treatment cycle, especially 8 weeks).
59. An antibody or antigen binding fragment thereof for use according to any one of paragraphs 1 to 55, wherein said antibody or binding fragment thereof is administered once approximately every three weeks, (in particular a single treatment cycle, especially 8 weeks).
60. An antibody or antigen binding fragment thereof for use according to any one of paragraphs 1 to 55, wherein the antibody or binding fragment thereof is administered once approximately every four weeks (for example monthly), (in particular a single treatment cycle, especially 8 weeks).
61. An antibody or antigen binding fragment thereof for use according to any one of the preceding paragraphs, wherein a loading dose in the range 400 to 900 mg, for example 400, 500, 600, 700, 800 or 900 mg is employed before administration of the treatment cycle.
62. An antibody or antigen binding fragment thereof for use according to any one of paragraphs 1 to 61, wherein a loading dose of 600 mg is employed before administration of the therapeutic doses.
63. An antibody or antigen binding fragment thereof for use according to paragraphs 61 or 62, wherein the loading dose is administered for 1 to 3 weeks, such as 1, 2 or 3 weeks prior to administration of the therapeutic doses.
64. An antibody or antigen binding fragment thereof for use according to any one of paragraphs 61 to 63, wherein the loading dose is administered only on week 0, for example 600 mg on week 0.
65. An antibody or antigen binding fragment thereof for use according to any one of paragraphs 61 to 63, wherein the loading dose is administered on weeks 0 and 1, for example 600 mg on week 0 and 600 mg on week 1.
66. An antibody or antigen binding fragment thereof for use according to any one of paragraphs 61 to 65, wherein the loading dose is administered on weeks 0, 1 and 2, for example 600 mg on week 0, 600 mg on week 1 and 600 mg on week 2
67. An antibody or antigen binding fragment thereof for use according to any one of paragraphs 1 to 60, wherein the treatment does not comprise a loading dose.
68. An antibody or antigen binding fragment thereof for use according to any one of the preceding paragraphs, wherein the dose is 200 mg.
69. An antibody or antigen binding fragment thereof for use according to any one of paragraphs 1 to 67, wherein the dose is 300 mg, for example administered 2 weekly.
70. An antibody or antigen binding fragment thereof for use according to any one of paragraphs 1 to 67, wherein the dose is 400 mg, for example administered 2 weekly or monthly.
71. An antibody or antigen binding fragment thereof for use according to any one of paragraphs 1 to 67, wherein the dose is 600 mg, for example administered monthly.
72. An antibody or antigen binding fragment thereof for use according to any one of paragraphs 1 to 71, wherein the therapeutic dose of the anti-IL13R antibody or binding fragment thereof is 400 mg dosed every 2 weeks (400 mg Q2W).
73. An antibody or antigen binding fragment thereof for use according to any one of paragraphs 1 to 71, wherein the therapeutic dose of the anti-IL13R antibody or binding fragment thereof is 600 mg dosed every 2 weeks (600 mg Q2W).
74. An antibody or antigen binding fragment thereof for use according to any one of paragraphs 1 to 71, wherein the therapeutic dose of the anti-IL13R antibody or binding fragment thereof is 300 mg dosed every 2 weeks (300 mg Q2W).
75. An antibody or antigen binding fragment thereof for use according to any one of paragraphs 1 to 71, wherein a loading dose of 600 mg is administered during weeks 0 and 1, followed by therapeutic dosing of 300 mg dosed every 2 weeks (300 mg Q2W).
76. An antibody or antigen binding fragment thereof for use according to any one of paragraphs 1 to 71, wherein a loading dose of 600 mg is administered during weeks 0 and 1, followed by therapeutic dosing of 400 mg dosed every 2 weeks (400 mg Q2W).
77. An antibody or antigen binding fragment thereof for use according to any one of paragraphs 1 to 71, wherein a loading dose of 600 mg is administered during weeks 0, 1 and 2 followed by therapeutic dosing of 600 mg dosed every 4 weeks (400 mg Q4W).
78. An antibody or antigen binding fragment thereof for use according to any one of the preceding paragraphs, wherein the treatment cycles comprises, a first dose at 600 mg, followed by three weekly doses of 400 mg, for example wherein the treatment cycle is repeated twice i.e. two treatment cycles lasting 8 weeks, in particular day 1 600 mg, approximately day 8 400 mg, approximately day 15 400 mg, approximately day 22 400 mg, approximately day 29 600 mg, approximately day 36 400 mg, approximately day 43 400 mg, and approximately day 50 400 mg are administered.
79. An antibody or antigen binding fragment thereof for use according to any one of the preceding paragraphs, wherein the disease modification occurs by day 8, wherein day 1 is the first administration of the antibody or binding fragment thereof.
80. An antibody or antigen binding fragment thereof for use according to any one of the preceding paragraphs, wherein the disease modification is a reduction in the SD score of POEM, for example wherein the reduction is a percentage from base line in the range −50 to −100%.
81. An antibody or antigen binding fragment thereof for use according to paragraph 80, wherein the disease modification in the range −50 to −100% is achieved by about day 57 following first administration on day 1, for example maximum disease modification is achieved by about day 57.
82. An antibody or antigen binding fragment thereof for use according to any one of the preceding paragraphs, wherein the antibody or antigen binding fragment binds an epitope FFYQ (for example same epitope as the antibody with a VH shown in SEQ ID NO: 51 and a VL shown in SEQ ID NO: 53, or a sequence at least 95% identical to any one of the same.
83. An antibody or antigen binding fragment thereof for use according to any one of the preceding paragraphs, wherein the antibody or antigen binding fragment thereof is an anti-IL13Rα1 antibody or antigen binding fragment thereof.
84. An antibody or antigen binding fragment thereof for use according to any one of the preceding paragraphs, wherein the antibody or antigen binding fragment thereof comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10.
85. An antibody or antigen binding fragment thereof for use according to any one of the preceding paragraphs, wherein the antibody or antigen binding fragment thereof comprises a VH domain comprising an amino acid sequence shown in SEQ ID NO: 51 or a sequence at least 95% identical thereto, in particular SEQ ID NO: 51.
86. An antibody or antigen binding fragment thereof for use according to any one of the preceding paragraphs, wherein the antibody or antigen binding fragment thereof comprises a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.
87. An antibody or antigen binding fragment thereof for use according to any one of the preceding paragraphs, wherein the antibody or antigen binding fragment thereof comprises a VL domain comprising an amino acid sequence shown in SEQ ID NO: 53 or a sequence at least 95% identical thereto, in particular SEQ ID NO: 53.
88. An antibody or antigen binding fragment thereof for use according to any one of the preceding paragraphs, wherein the antibody comprises a VL domain comprising an amino acid sequence shown in SEQ ID NO: 53 or a sequence at least 95% identical thereto, in particular SEQ ID NO: 53, and a VH comprising an amino acid sequence shown in SEQ ID NO: 51 or a sequence at least 95% identical thereto, in particular SEQ ID NO: 51.
89. A pharmaceutical formulation comprising the antibody or binding fragment thereof for use according to any one of paragraphs 1 to 88, said formulation comprising 150 to 210 mg/ml of an anti-IL-13R antibody or antigen binding fragment thereof, for example 150, 155, 160, 165, 170, 175, 180, 185, 190, 195, 200, 205 or 210 mg/ml, in particular 150 mg/ml, 175 mg/ml or 200 mg/ml;
   170 to 250 mM of arginine (such as Arg-HCl or Arg-Glu), for example 170, 175, 180, 185, 190, 195, 200, 205, 210, 215, 220, 225, 230, 235, 240, 245 or 250 mM, in particular 150 mM, 175 mM or 250 mM;
   20 to 50 mM histidine buffer, for example 20, 25, 30, 35, 40, 45 or 50 mM, such as 20 mM or 50 mM histidine buffer;
   0.01-0.03% of a non-ionic surfactant, such as 0.02% w/w; and
   wherein the pH of the formulation is in the range 6.0 to 7.0, such as 6.0, 6.1, 6.2, 6.3, 6.4, 6.5, 6.6, 6.7, 6.8, 6.9 or 7.0, in particular 6.5.
90. A pharmaceutical formulation comprising an antibody or binding fragment thereof for use according to any one of paragraphs 1 to 88, said formulation comprising 175 to 250 mg/ml of an anti-IL-13R antibody or antigen binding fragment thereof, for example 175, 180, 185, 190, 195, 200, 205, 210, 215, 220 or 225 mg/ml, in particular 200 mg/ml, 225 mg/ml or 250 mg/ml;
   15 to 75 mM of tryptophan, such as 15 to 60 mM, in particular 25 to 50 mM tryptophan; 190 to 270 mM of arginine (such as Arg-HCl or Arg-Glu), for example 190, 195, 200, 205, 210, 215, 220, 225, 230, 235, 240, 245, 250, 255, 260, 265 or 270 mM, in particular 200, 210, 225, 235, 250 or 260 mM;
   0.01-0.03% of a non-ionic surfactant, for example 0.01-0.03% w/w, such as 0.02% w/w; a buffer (such as histidine buffer); and
   wherein the pH of the formulation is in the range 6.0 to 7.0, such as 6.0, 6.1, 6.2, 6.3, 6.4, 6.5, 6.6, 6.7, 6.8, 6.9 or 7.0, in particular 6.4.
91. A pharmaceutical formulation comprising an antibody or binding fragment thereof for use according to any one of paragraphs 1 to 88, said formulation comprising 175 to 250 mg/ml of an anti-IL-13R antibody or antigen binding fragment thereof, for example 175, 180, 185, 190, 195, 200, 205, 210, 215, 220, 225, 230, 235, 240, 245 or 250 mg/ml, in particular 190, 200 mg/ml, 210 mg/ml, 225 mg/ml or 250 mg/ml, such as 200 mg/ml of an anti-IL-13R antibody or antigen binding fragment thereof;
   5 to 100 mM of tryptophan (including 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mM), for example 15 to 75 mM of tryptophan, such as 15 to 60 mM, in particular 25 to 50 mM tryptophan, such as 20 mM, 50 or 80 mM tryptophan;
   140 to 290 mM of arginine (including 150 or 151 to 290 nM), for example 160 to 290 mM of arginine (such as Arg-HCl or Arg-Glu), for example 160, 165, 175, 180, 185, 190, 195, 200, 205, 210, 215, 220, 225, 230, 235, 240, 245, 250, 255, 260, 265, 270, 275, 280 285 or 290 mM, in particular 150, 185, 215, 225, 250, 260 or 280 mM arginine;
   0.01-0.03% of a non-ionic surfactant (such as polysorbate), for example 0.01-0.03% w/w, such as 0.01%, 0.015%, 0.020%, 0.025% or 0.030% in particular 0.02% w/w of a non-ionic surfactant;
   a buffer (such as histidine buffer), for example 15 to 55 mM of a buffer (including 25 or 26 to 55), such as 15, 20, 25, 30, 35, 40, 45, 50 or 55 mM of a histidine buffer; in particular 20, 35 or 50 mM histidine buffer; and
   wherein the pH of the formulation is in the range 5.5 to 7.2 (including 5.5, 5.6, 5.7, 5.8, 5.9, 6.0, 6.1, 6.2, 6.3, 6.4, 6.5, 6.6, 6.7, 6.8, 6.9 or 7.0, for example 6.0 to 7.0, such as 6.0, 6.1, 6.2, 6.3, 6.4, 6.5, 6.6, 6.7, 6.8, 6.9 or 7.0, in particular 5.8, 6.3, 6.4, 6.5, 6.6, 6.7 or 6.8.
92. A method of treating a patient having sleep loss or sleep disturbance comprising administering an antibody or antigen binding fragment thereof, which is an inhibitor of signalling through of the IL-13Rα1 by binding the said receptor, or a pharmaceutical formulation comprising the same, for example according to any one of paragraphs 1 to 88, by parenteral administration of a treatment cycle comprising a dose in the range 200 mg to 600 mg, (for example 300, 400, 500 or 600 mg such as 400 to 600 mg), for example wherein the treatment results in a reduction in the sleep disturbance (SD) score of the Patient Oriented Eczema Measure (POEM), such as in the range −50 to −100% from the baseline, for example a reduction of 2 to 4 points from the baseline.
93. Use of an antibody or antigen binding fragment thereof, which is an inhibitor of signalling through of the IL-13Rα1 by binding the said receptor, or a pharmaceutical formulation comprising the same, for example according to any one of paragraphs 1 to 88, in the manufacture of a medicament for the treatment of sleep loss or sleep disturbance in a patient by parenteral administration of a treatment cycle comprising a dose in the range 200 mg to 600 mg, (for example 300, 400, 500 or 600 mg such as 400 to 600 mg), for example wherein the treatment results in a reduction in the sleep disturbance (SD) score of the Patient Oriented Eczema Measure (POEM), for example in the range −50 to −100% from the baseline, such as a reduction of 2 to 4 points from the baseline.

Surprisingly, the present inventors have established that the symptoms and severity of sleep loss or sleep disturbance can be treated and/or ameliorated by administering an anti-IL13R antibody or antigen binding fragment, such as eblasakimab to subjects in need thereof.

In one embodiment there is provided a method of treating a patient having sleep loss or sleep disturbance, such as a highly allergic patient as defined herein with an antibody or binding fragment thereof or a pharmaceutical formulation as defined herein.

In one embodiment there is provided use of an antibody or binding fragment thereof or a pharmaceutical formulation, as defined herein for the manufacture of a medicament for the treatment of sleep loss or sleep disturbance in a patient, for example in a highly allergic patient.

In one embodiment the improvement in sleep is independent of improvement in the underlying disease (for example disclosed herein), such as allergy.

In one embodiment the improvement in sleep is associated with improvement in the underlying disease (for example disclosed herein), such as allergy.

In one embodiment treatment with eblasakimab reduces the overall burden of disease, for example reduces the overall burden of sleep loss or sleep disturbance in a patient.

In one embodiment treatment with eblasakimab improves quality of life in a patient having sleep loss or sleep disturbance, for example a highly allergic patient.

In one embodiment the patient has a type 2-driven inflammatory skin disorder, for example a skin disorder exacerbated by pro-inflammatory cytokines present in the skin. Thus, in one embodiment eblasakimab is suitable for treating a patient having a type-2 driven inflammatory skin disorder. In one embodiment the type 2-driven inflammatory skin disorder is atopic dermatitis (AD).

In one embodiment, the patient has pruritis. In one embodiment itch signalling in the patient is exacerbated by pro-inflammatory cytokines present in the skin of the patient, which may cause an immune response that disrupts the skin barrier and drives disease pathology. Thus, in one embodiment eblasakimab is able to reduce or inhibit itch signalling in a patient. In one embodiment treatment with eblasakimab prevents or reduces the disruption of the skin barrier in a patient. In one embodiment eblasakimab dampens or inhibits the immune response that drives disease pathology in a patient having pruritus.

Whilst not wishing to be bound by theory eblasakimab is able to inhibit the signalling pathways for pruritis and/or inhibit signalling for neuropathic pain (such as peripheral and./or central neuropathic pain, in particular peripheral neuropathic pain) thereby leading to improved sleep.

In one embodiment the pro-inflammatory cytokines are IL-13, IL-4 or a combination of both IL-13 and IL4. In one embodiment IL-13, IL-4 or both act as neuronal enhancers for the amplification of itch pathways through the 13Rα1 subunit of the Type-2 receptor. Thus, in one embodiment eblasakimab inhibits or dampens neuronal enhancers for the amplification of itch pathways, such as IL-13 and/or IL-4.

In one embodiment treatment with eblasakimab results in a reduction of pruritic neuronal response, for example via eblaskimab's dual blockade of both IL-4 and IL-13 through the Type 2 receptor.

Improved sleep has significant health benefits, including improved mood, improved ability to concentrate, reduced generic inflammatory markers in the body, increased energy, reduced levels of obesity, reduced risk of heart disease, reduced risk of diabetes, improvements in relationships, improvements in self-esteem, improved immune system, improved ability to deal with stress.

In one embodiment a combination therapy is employed comprising the antibody, antigen binding fragment thereof or a formulation according to the present disclosure and a further medicament.

In one embodiment the further medicament is for the treatment of sleep loss, for example sleeping aids, including but not limited to melatonin, zolpidem, zaleplon, eszopiclone, ramelteon, suvorexant, lamborexant or doxepain. In one embodiment, the further medicament is for treating the symptoms of sleep loss such as narcolepsy. Examples of such medicaments include but not limited to stimulants or wake-promoting medicaments, for example modafinil, armodafinil, pitolisant or solriamfetol.

Surprisingly disease modification following treatment with an anti-IL-13Rα1 antibody or antigen binding fragment thereof according to the present disclosure closely follows reduction in TARC, in fact the TARC reduction and EASI reduction correlate closely.

In one embodiment the patient has a skin condition, for example selected from the group comprising dermatitis; such as atopic dermatitis (AD), contact dermatitis, neurodermatitis or seborrheic dermatitis; eczema; hand-foot-and-mouth disease; hives (including urticaria associated with Lupus), psoriasis, an infection, such as a fungal or bacterial infection, for example impetigo or folliculitis; an allergic skin reaction; Ehlers-Danlos syndrome; asthma; and angioedema, such as hereditary angioedema (HAE).

In one embodiment the patient has dermatitis, for example atopic dermatitis (AD), contact dermatitis, neurodermatitis or seborrheic dermatitis. In one embodiment the patient has atopic dermatitis (AD), for example moderate to severe atopic dermatitis. In one embodiment the patient has eczema. In one embodiment, the sleep loss or sleep disturbance is due to the patient's eczema. In one embodiment the patient has hives. In one embodiment the patient has Ehlers-Danlos syndrome (EDS), asthma or angioedema (such as HAE). In one embodiment the patient has Ehlers-Danlos syndrome. In one embodiment the patient has asthma. In one embodiment the patient has angioedema, such as HAE.

The present inventors have also established that the antibodies, antigen binding fragments and compositions of present invention can be employed to treated other diseases with an allergic component, for example allergic epithelial disease, such as allergic asthma, and eosinophilic esophagitis.

Thus, in an independent aspect there is provided treatment of an allergic disease, for example with elevated IgE levels (elevated in comparison to normal levels), with an antibody, binding fragment or composition comprising the same, which is an inhibitor of signalling through IL-13Rα1, by binding the said receptor, for example as described elsewhere herein (for atopic dermatitis).

Whilst not wishing to be bound by theory, the present inventors believe that the same cytokines and similar or corresponding mechanisms are at work in these allergic diseases as in this population of atopic dermatitis.

In one embodiment the allergic disease is manifested in epithelial tissue.

In one embodiment the allergic disease is allergic asthma, for example poorly controlled and/or moderate to severe asthma. In one embodiment the allergic disease is eosinophilic esophagitis, for example poorly controlled and/or moderate to severe eosinophilic esophagitis.

In one embodiment the treatment of the present disclosure is given to a patient with an acute allergic reaction.

In one embodiment the patient is identified as allergic before treatment, for example where the baseline has been established and is at a level of at least 10,000 KU/L+/−2,000.

Preferences described herein for atopic dermatitis, like dose and the like apply equally to allergic disease indications.

In one embodiment the allergic disease is not atopic dermatitis. In one embodiment the allergic disease is not eosinophilic esophagitis. In one embodiment the allergic disease is not psoriasis.

### DETAILED DISCLOSURE

Sleep loss, sleep deprivation or sleep disturbance as used herein refers to an inability to fall asleep or to stay asleep, for example due to disruptions to the sleep-wake cycle. In one embodiment patients wake up at one or more times during the night, for example on average 1, 2, 3, 4, 5 or more times per night. In one embodiment patients on average take at least 45 minutes to fall to sleep, for example 1, 1.5, 2 hours to fall asleep. In one embodiment patients on average are awake for at least 30 minutes in the night (such as 1, 1.5, 2 hours or more), for example at 3 times per week. In one embodiment patients wake up at one or more times during the night, for example on average 1, 2, 3, 4, 5 or more times per night and on average take at least 45 minutes to fall to sleep, for example 1, 1.5, 2 hours to fall asleep. In one embodiment patients wake up at one or more times during the night, for example on average 1, 2, 3, 4, 5 or more times per night and average are awake for at least 30 minutes in the night (such as 1, 1.5, 2 hours or more), for example at 3 times per week. In one embodiment patients on average take at least 45 minutes to fall to sleep, for example 1, 1.5, 2 hours to fall asleep and on average are awake for at least 30 minutes in the night (such as 1, 1.5, 2 hours or more), for example at 3 times per week. In one embodiment patients wake up at one or more times during the night, for example on average 1, 2, 3, 4, 5 or more times per night and on average take at least 45 minutes to fall to sleep, for example 1, 1.5, 2 hours to fall asleep and on average are awake for at least 30 minutes in the night (such as 1, 1.5, 2 hours or more), for example at 3 times per week.

Patient Oriented Eczema Measure (POEM) as used herein refers to a 7-item questionnaire for patients that assesses presence of disease symptoms (dryness, itching, flaking, cracking, sleep loss, bleeding, and weeping) over the last week using a scoring system of 0 (no days) to 4 (every day). The total score ranges from 0 to 28 with higher scores indicating greater intensity of eczema.

Sleep disturbance (SD) score as used herein refers to the score for the question in the POEM questionnaire relating to sleep disturbance. The score ranges from 0 (no days of sleep disturbance) to 4 (sleep disturbance every day). The relevant question is typically worded as follows: over the last week, on how many nights has your sleep been disturbed because of your eczema? The available answers are for example: no days (SD score 0), 1-2 days (SD score 1), 3-4 days (SD score 2), 5-6 days (SD score 3) or everyday (SD score 4).

SCORAD as used herein refers to a clinical tool used to assess the extent and severity of atopic dermatitis (SCORing Atopic Dermatitis). Within the context of the present disclosure, SCORAD is an alternative scoring tool that may be used instead of POEM.

To calculate the SCORAD index, first the extent of disease is determined using the rule of nines to estimate percentage of the affected body surface area. The maximum value for the extent of the disease is 100%. Next, disease severity is then calculated based on six characteristics: erythema, edema, oozing/crusts, excoriations, lichenification, and dryness. Each characteristic is given a score between 0 and 3, where 0 is absent and 3 is severe. The scores for each characteristic are added together for a total severity score of up to 18. Subjective symptoms including sleep loss and pruritus are measured using a 10 cm visual analogue scale with a total maximum score of 20. Finally, the SCORAD index is then calculated with the following formula: Extent/5+7xSeverity/2+Subjective symptoms.

The sleep deprivation component of SCORAD as used herein refers to the score for the sleep loss question of the SCORAD. The score reflects an average of the last 3 days or nights experienced by the patient and ranges from 0 (no sleep loss) to 10 (worst imaginable sleep loss).

Sleep disturbance numerical rating scale (SD NRS) as used herein refers to a single item patient reported outcome (PRO) measure for quantifying sleep disturbance/sleep loss. The SD NRS asks patients to score their sleep loss during the previous night on a scale of 0 (“no sleep loss”) to 10 (“I did not sleep at all”).

In one embodiment the reduction in SD score during or after treatment is in the range of −50% to −100% from the baseline, for example −50%, −55%, −60%, −65%, −70%, −75%, −80%, −85%, −90%, −95% or −100%. In one embodiment the reduction in SD score during or after is in the range of −55% to −100% from the baseline, for example −55%, −60%, −65%, −70%, −75%, −80%, −85%, −90%, −95% or −100%. In one embodiment the reduction in SD score during or after is in the range of −60% to −100% from the baseline, for example −60%, −65%, −70%, −75%, −80%, −85%, −90%, −95% or −100%. In one embodiment the reduction in SD score during or after is in the range of −65% to −100% from the baseline, for example −65%, −70%, −75%, −80%, −85%, −90%, −95% or −100%. In one embodiment the reduction in SD score during or after is in the range of −70% to −100% from the baseline, for example −70%, −75%, −80%, −85%, −90%, −95% or −100%. In one embodiment the reduction in SD score during or after is in the range of −75% to −100%, for example −75%, −80%, −85%, −90%, −95% or −100%. In one embodiment the reduction in SD score during or after is in the range of −80% to −100% from the baseline, for example −80%, −85%, −90%, −95% or −100%. In one embodiment the reduction in SD score during or after is in the range of −85% to −100% from the baseline, for example −85%, −90%, −95% or −100%. In one embodiment the reduction in SD score during or after is in the range of −90% to −100% from the baseline, for example −90%, −95% or −100%. In one embodiment the reduction in SD score during or after is in the range of −95% to −100% from the baseline, for example −95% or −100%. In one embodiment the reduction in SD score during or after is in the range of −15% to −25%, for example −15%, −16%, −17%, −18%, −19%, −20%, −21%, −22%, −23%, −24% or −25%. In one embodiment the reduction in SD score during or after is in the range of −25% to −35% from the baseline, for example −25%, −26%, −27%, −28%, −29%, −30%, −31%, −32%, −33%, −34% or −35%. In one embodiment the reduction in SD score during or after is in the range of −35% to −45% from the baseline, for example −35%, −36%, −37%, −38%, −39%, −40%, −41%, −42%, −43%, −44% or −45%. In one embodiment the reduction in SD score during or after is in the range of 45% to −55% from the baseline, for example −45%, −46%, −47%, −48%, −49%, −50%, −51%, −52%, −53%, −54% or −55%. In one embodiment the reduction in SD score during or after is in the range of 55% to −65% from the baseline, for example −55%, −56%, −57%, −58%, −59%, −60%, −61%, −62%, −63%, −64% or −65%. In one embodiment the reduction in SD score during or after is in the range of 65% to −75% from the baseline, for example −65%, −66%, −67%, −68%, −69%, −70%, −71%, −72%, −73%, −74% or −75%. In one embodiment the reduction in SD score during or after is in the range of 75% to −85% from the baseline, for example −75%, −76%, −77%, −78%, −79%, −80%, −81%, −82%, −83%, −84% or −85%. In one embodiment the reduction in SD score during or after is in the range of 85% to −95% from the baseline, for example −85%, −86%, −87%, −88%, −89%, −90%, −91%, −92%, −93%, −94% or −95%. In one embodiment the reduction in SD score during or after is in the range of 95% to −100% from the baseline, for example −95%, −96%, −97%, −98%, −99%, or −100%.

In one embodiment, the reduction in SD score during or after treatment is 1 to 4 points from the baseline, such as 1, 2, 3 or 4 points. In one embodiment, the reduction in SD score during or after treatment is 2 or more points from the baseline, such as 2, 3, or 4 points. In one embodiment, the reduction in SD score during or after treatment is 3 or more points from the baseline, such as 3 or 4 points.

In one embodiment, the reduction in the sleep deprivation component of SCORAD during or after treatment is in the range of −20% to −100% from the baseline, for example −20%, −25%, 30%, −35%, −40%, −45%, −50%, −55%, −60%, −65%, −70%, −75%, −80%, −85%, −90%, −95% or −100%. In one embodiment the reduction in the sleep deprivation component of SCORAD during or after treatment is in the range of −30% to −100% from the baseline, for example 30%, −35%, −40%, −45%, −50%, −55%, −60%, −65%, −70%, −75%, −80%, −85%, −90%, −95% or −100%. In one embodiment, the reduction in the sleep deprivation component of SCORAD during or after treatment is in the range of −40% to −100% from the baseline, for example −40%, −45%, −50%, −55%, −60%, −65%, −70%, −75%, −80%, −85%, −90%, −95% or −100%. In one embodiment, the reduction in the sleep loss or sleep deprivation component of SCORAD during or after treatment is in the range of −50% to −100% from the baseline, for example −50%, −55%, −60%, −65%, −70%, −75%, −80%, −85%, −90%, −95% or −100%. In one embodiment, the reduction in the sleep deprivation component of SCORAD during or after treatment is in the range of −60% to −100% from the baseline, for example −60%, −65%, −70%, −75%, −80%, −85%, −90%, −95% or −100%. In one embodiment, the reduction in the sleep deprivation component of SCORAD during or after treatment is in the range of −70% to −100% from the baseline, for example −−70%, −75%, −80%, −85%, −90%, −95% or −100%. In one embodiment, the reduction in the sleep deprivation component of SCORAD during or after treatment is in the range of −80% to −100%, for example −80%, −85%, −90%, −95% or −100%. In one embodiment, the reduction in the sleep deprivation component of SCORAD during or after treatment is in the range of −20% to −70% from the baseline, such as −20%, −25%, −30%, −35%, −40%, −50%, −55%, −60%, −65%, or −70%. In one embodiment, the reduction in the sleep deprivation component of SCORAD during or after treatment is in the range of −30% to −60% from the baseline, such as −30%, −35%, −40%, −50%, −55% or −60%. In one embodiment, the reduction in the sleep deprivation component of SCORAD during or after treatment is in the range of −35% to −55% from the baseline, such as −35%, −−40%, −50%, or −55%.

In one embodiment, the reduction in the sleep deprivation component of SCORAD is 1 to 10 points from the baseline, such as 1, 2, 3, 4, 5, 6, 7, 8, 9 or 10 points. In one embodiment, the reduction in the sleep deprivation component of SCORAD is 2 or more points from the baseline, such as 2, 3, 4, 5, 6, 7, 8, 9 or 10 points. In one embodiment, the reduction in the sleep deprivation of SCORAD is 3 or more points from the baseline, such as 3, 4, 5, 6, 7, 8, 9 or 10 points. In one embodiment, the reduction in the sleep deprivation of SCORAD is 4 or more points from the baseline, such as 4, 5, 6, 7, 8, 9 or 10 points. In one embodiment, the reduction in the sleep deprivation of SCORAD is 5 or more points from the baseline, such as 5, 6, 7, 8, 9 or 10 points. In one embodiment, the reduction in the sleep deprivation of SCORAD is 6 or more points from the baseline, such as 6, 7, 8, 9 or 10 points. In one embodiment, the reduction in the sleep deprivation of SCORAD is 7 or more points from the baseline, such as 7, 8, 9 or 10 points. In one embodiment, the reduction in the sleep deprivation of SCORAD is 8 or more points from the baseline, such as 8, 9 or 10 points. In one embodiment, the reduction in the sleep deprivation of SCORAD is 9 or more points from the baseline, such as 9 or 10 points.

In one embodiment, the reduction in the sleep disturbance numerical rating scale (SD NRS) during or after treatment is in the range of −20% to −100% from the baseline, for example −20%, −25%, 30%, −35%, −40%, −45%, −50%, −55%, −60%, −65%, −70%, −75%, −80%, −85%, −90%, −95% or −100%. In one embodiment the reduction in the SD NRS during or after treatment is in the range of −30% to −100% from the baseline, for example 30%, −35%, −40%, −45%, −50%, −55%, −60%, −65%, −70%, −75%, −80%, −85%, −90%, −95% or −100%. In one embodiment, the reduction in SD NRS during or after is in the range of −40% to −100% from the baseline, for example −40%, −45%, −50%, −55%, −60%, −65%, −70%, −75%, −80%, −85%, −90%, −95% or −100%. In one embodiment, the reduction in SD NRS during or after is in the range of −50% to −100% from the baseline, for example−50%, −55%, −60%, −65%, −70%, −75%, −80%, −85%, −90%, −95% or −100%. In one embodiment, the reduction in SD NRS during or after is in the range of −60% to −100% from the baseline, for example −60%, −65%, −70%, −75%, −80%, −85%, −90%, −95% or −100%. In one embodiment, the reduction in the SD NRS during or after is in the range of −70% to −100% from the baseline, for example −70%, −75%, −80%, −85%, −90%, −95% or −100%. In one embodiment, the reduction in the SD NRS during or after is in the range of −80% to −100% from the baseline, for example −80%, −85%, −90%, −95% or −100%. In one embodiment, the reduction in SD NRS during or after is in the range of −20% to −70% from the baseline, such as −20%, −25%, −30%, −35%, −40%, −50%, −55%, −60%, −65%, or −70%. In one embodiment, the reduction in SD NRS during or after is in the range of −30% to −60% from the baseline, such as −30%, −35%, −40%, −50%, −55% or −60%. In one embodiment, the reduction in SD NRS during or after is in the range of −35% to −55% from the baseline, such as −35%, −40%, −50%, or −55%.

In one embodiment, the reduction in SD NRS is 1 to 10 points from the baseline, such as 1, 2, 3, 4, 5, 6, 7, 8, 9 or 10 points. In one embodiment, the reduction in SD NRS is 2 or more points from the baseline, such as 2, 3, 4, 5, 6, 7, 8, 9 or 10 points. In one embodiment, the reduction in SD NRS is 3 or more points from the baseline, such as 3, 4, 5, 6, 7, 8, 9 or 10 points. In one embodiment, the reduction in SD NRS is 4 or more points from the baseline, such as 4, 5, 6, 7, 8, 9 or 10 points. In one embodiment, the reduction in SD NRS is 5 or more points from the baseline, such as 5, 6, 7, 8, 9 or 10 points. In one embodiment, the reduction in SD NRS is 6 or more points from the baseline, such as 6, 7, 8, 9 or 10 points. In one embodiment, the reduction in SD NRS is 7 or more points from the baseline, such as 7, 8, 9 or 10 points. In one embodiment, the reduction in SD NRS is 8 or more points from the baseline, such as 8, 9 or 10 points. In one embodiment, the reduction in SD NRS is 9 or more points from the baseline, such as 9 or 10 points.

In one embodiment, the patient has a baseline SD score of at least 1, such as 1, 2, 3 or 4. In one embodiment, the patient has a baseline SD score of at least 2, such as 2, 3 or 4. In one embodiment, the patient has a baseline SD score of at least 3, such as 3 or 4. In one embodiment, the patient has a baseline SD score of 4.

In one embodiment, the patient has a baseline sleep deprivation component of SCORAD of at least 1 point, such as 1, 2, 3, 4, 5, 6, 7, 8, 9 or 10 points. In one embodiment, the patient has a baseline sleep deprivation component of SCORAD of at least 2 points, such as 2, 3, 4, 5, 6, 7, 8, 9 or 10 points. In one embodiment, the patient has a baseline sleep deprivation component of SCORAD of at least 3 points, such as 3, 4, 5, 6, 7, 8, 9 or 10 points. In one embodiment, the patient has a baseline sleep deprivation component of SCORAD of at least 4 points, such as 4, 5, 6, 7, 8, 9 or 10 points. In one embodiment, the patient has a baseline sleep deprivation component of SCORAD of at least 5 points, such as 5, 6, 7, 8, 9 or 10 points. In one embodiment, the patient has a baseline sleep deprivation component of SCORAD of at least 6 points, such as 6, 7, 8, 9 or 10 points. In one embodiment, the patient has a baseline sleep deprivation component of SCORAD of at least 7 points, such as 7, 8, 9 or 10 points. In one embodiment, the patient has a baseline sleep deprivation component of SCORAD of at least 8 points, such as 8, 9 or 10 points. In one embodiment, the patient has a baseline sleep deprivation component of SCORAD of at least 9 points, such as 9 or 10 points.

In one embodiment, the patient has a baseline SD NRS of at least 1 point, such as 1, 2, 3, 4, 5, 6, 7, 8, 9 or 10 points. In one embodiment, the patient has a baseline SD NRS of at least 2 points, such as 2, 3, 4, 5, 6, 7, 8, 9 or 10 points. In one embodiment, the patient has a baseline SD NRS of at least 3 points, such as 3, 4, 5, 6, 7, 8, 9 or 10 points. In one embodiment, the patient has a baseline SD NRS of at least 4 points, such as 4, 5, 6, 7, 8, 9 or 10 points. In one embodiment, the patient has a baseline SD NRS of at least 5 points, such as 5, 6, 7, 8, 9 or 10 points. In one embodiment, the patient has a baseline SD NRS of at least 6 points, such as 6, 7, 8, 9 or 10 points. In one embodiment, the patient has a SD NRS of at least 7 points, such as 7, 8, 9 or 10 points. In one embodiment, the patient has a baseline SD NRS of at least 8 points, such as 8, 9 or 10 points. In one embodiment, the patient has a baseline SD NRS of at least 9 points, such as 9 or 10 points.

Disease modification as employed herein relates to improvements in the disease status for example as measured by a clinically relevant score, in particular a reduction in the sleep disturbance score of POEM. Thus, in one embodiment, the disease modification is a reduction in the sleep disturbance score of POEM. In another embodiment, the disease modification is a reduction in the sleep deprivation component of SCORAD. In another embodiment, the disease modification is a reduction in the sleep disturbance numerical rating scale(SD NRS).

A clinically relevant score is a score used in the clinic, for example used by a physician.

Pruritus or pruritis as used herein refers to a condition where the skin becomes itchy. It results in a very uncomfortable and irritating sensation that compels the individual to scratch the skin. Pruritus can be caused by a range of different conditions, including skin conditions (such as atopic dermatitis and psoriasis), internal diseases (such as liver and kidney disease, lymphoma), nerve disorders (such as multiple sclerosis), psychiatric conditions (such as anxiety and obsessive compulsive disorder), and allergic reactions.

Pruritus numerical score/scale (P-NRS) as used herein is a patient assessed score of the intensity of his or her itch over the previous 24 hours based on a scale of 0 to 10, with a score of 0 referring to “no itch” and a top score of 10 referring to “worst imaginable itch”. Average pruritus numerical score/scale (average P-NRS) as used herein refers a patient assessed score of the average intensity of his or her itch over the previous 24 hours based on a scale of 0 to 10, with a score of 0 referring to “no itch” and a top score of 10 referring to “worst imaginable itch”. Peak pruritus numerical score/scale (peak P-NRS) or worst pruritus numerical score/scale (worst P-NRS) as used herein refers a patient assessed score of the worst intensity of his or her itch over the previous 24 hours based on a scale of 0 to 10, with a score of 0 referring to “no itch” and a top score of 10 referring to “worst imaginable itch”.

In one embodiment the disease is modified by a percentage reduction in Eczema Area and Severity Index (EASI) score in the range −20 to −100% from the baseline, such as EASI 50, EASI 75 or EASI 90. EASI score and EASI are used interchangeably herein.

Eczema Area and Severity Index (EASI) score as used herein is a tool used to measure the area (which indicates the extent of disease) and severity of atopic eczema. The number after the term “EASI” indicates the % decrease in the score from baseline. Thus, EASI 50 for example refers to 50% decrease in the score and EASI 90 refers to a 90% decrease in the score.

In one embodiment disease modification is measured as a reduction in IGA.

In one embodiment there is a provided a reduction in the Investigator Global Assessment (IGA) with/after treatment according to the present disclosure, for example an assessment of 0, 1 or 2, (no inflammatory signs, almost clear and mild disease respectively). In particular there is provided an IGA score of 0 or 1.

Investigator's global assessment (IGA) as used herein refers to a tool for the assessment of atopic dermatitis. It uses a 0-5 point scale depending on the severity of a patient's symptoms:

| Score | Severity | Description of symptoms |
|---|---|---|
| 0 | clear | no inflammatory signs |
| 1 | almost clear | Just perceptible erythema and just perceptible |
| | | papulation/infiltration |
| 2 | mild disease | Mild erythema and mild papulation/infiltration |
| 3 | moderate disease | moderate erythema and moderate papulation/ |
| | | infiltration |
| 4 | severe disease | severe erythema and severe papulation/ |
| | | infiltration |
| 5 | very severe | severe erythema and severe papulation/ |
| | disease | infiltration with oozing/crusting |

In an independent aspect there is provided use of an antibody, antigen binding fragment or pharmaceutical formulation as disclosed herein for the treatment or amelioration of allergic food responses, in particular severe allergic food response, such as peanut allergy.

In one embodiment, the highly allergic patient's baseline IgE levels have been established and are at a level of at least at least 10,000 KU/L+/−2,000, such as 8000, 8500, 9000, 9500, 10000, 10500, 11000, 11500 or 12000 KU/L.

In one embodiment, the patient has been identified as a highly allergic patient before the treatment is administered, for example, wherein the patient's baseline IgE levels have been established and are at a level of at least 10,000 KU/L+/−2,000. Thus, in one embodiment the patient is identified as a highly allergic patient wherein the patient's baseline IgE levels have been established and are at a level of at least 10,000 KU/L+/−2,000, prior to administration of the antibody or binding fragment thereof, pharmaceutical formulation or medicament as defined herein. Accordingly, in one embodiment the target population to be treated is highly allergic patients whose baseline IgE levels have been established and are at a level of at least 10,000 KU/L 15+/−2,000.

In one embodiment the patient is identified as highly allergic by clinical observation.

Poorly controlled as employed herein refers to poorly controlled by existing approved medicaments, for example topical medicine and/or dupilumab. The results suggest that eblasakimab has a comparable or in some cases a higher efficacy compared to Dupilumab, thus demonstrating the potential of eblasakimab as an alternative therapy for the treatment and management of sleep loss.

Thus, in one embodiment there is provided treatment of a patient population where the patient had poorly controlled symptoms and/or adverse side effects when treated with dupilumab i.e. the patient population has failed with treatment on dupilumab. Accordingly, in one embodiment, the patient was previously administered with dupilumab. In one embodiment, the patient had sleep loss or sleep disturbance that was poorly controlled by dupilumab. In one embodiment, the patient has atopic dermatitis that was poorly controlled by dupilumab. In one embodiment, the patient has atopic dermatitis and had sleep loss or sleep disturbance that was poorly controlled by dupilumab.

Asthma as used herein is a respiratory disease characterized by inflammation and bronchospasm, wherein the muscles around the airways tighten and contract in an attempt to keep the airways open. This leaves patients with cough, wheezing, chest tightness and shortness of breath. When the breathing issues become severe, this is typically referred to as an asthma attack.

A typical asthma may be characterised by a dry cough or feeling of obstruction in the throat Allergic asthma (used interchangeably with allergy-induced asthma) as used herein refers to form of asthma whereby the lungs of a patient become inflamed, and the airways tighten in response to the inhalation of an allergen. Common allergens include pollen, dust, animal dander and mold. Patients with allergic asthma experience many of the same symptoms as patients with non-allergic asthma—cough, wheezing, chest tightness and shortness of breath. Hence, the major difference between the two conditions is that patients with allergic asthma normally experience symptoms after inhaling an allergen.

Eosinophilic esophagitis (EOE) as used herein refers to a chronic immune system disease where eosinophils build up in the esophagus (eosinophils are not normally found in the esophagus). This build-up occurs as a reaction to foods, allergens or acid reflux, and may inflame and cause injury to the esophageal tissue. This in turn may lead to narrowing of the esophagus and difficulty swallowing. In serious cases it may even result in medical emergencies due to food getting stuck in the throat. The majority of patients with EOE are atopic. Thus, individuals with atopic dermatitis, food or other environmental allergies are at greater risk of developing EOE. Family history of EOE is also a risk factor for the condition. No medications are currently approved by the US FDA for treating EOE. However, proton pump inhibitors (PPIs) have been demonstrated to reduce esophageal inflammation in some EOE patients and are thus often used as a first treatment. Corticosteroids may also be administered to help control inflammation.

Atopic dermatitis (AD) as used herein refers to inflammation of the skin and includes: dry/itchy skin and red rashes. AD can be a very painful, demoralising and psychologically damaging disease. The most common side effect of AD is pruritus, and a diagnosis of active AD cannot be made without an accompanying history of itching. Indeed, AD patients often complain that the pruritus is the most annoying and hardest symptom they have to cope with.

Psoriasis as used herein refers to a skin disorder that causes skin cells to multiply much faster than normal. This results in red, itchy scaly patches, which are typically found on the scalp, knees, elbows, and trunk. There are different categories of psoriasis, including pustular psoriasis, guttate psoriasis, inverse psoriasis and erythrodermic psoriasis. Most types of psoriasis go through cycles, flaring for a few weeks or months, then subsiding for a time or even going into remission.

Angioedema as used herein refers to the rapid edema (swelling) of the area beneath the skin or mucosa caused by the build-up of fluid, typically in response to an allergic trigger. Angioedema can affect different parts of the body, but it typically manifests in the eyes, lips, genitalia, hands and feet. Angioedema can arise with hives or alone. In more serious cases, angioedema can result in breathing difficulties, abdominal pain and dizziness.

Ehlers-Danlos syndrome (EDS) as used herein refers to a group of inherited disorders wherein the connective tissues, such as tendons, ligaments, blood vessels, etc are affected. There are 13 types of EDS, including hypermobile EDS (the most common), classical EDS, vascular EDS and kyphoscoliotic EDS. The different types of EDS share some common symptoms such as increased range of joint mobility (hypermobility), and stretchy and fragile skin. There are no known cures for EDS and treatment is largely supportive in nature, for example physiotherapy.

Hives (urticaria) as used herein refers to a sudden outbreak of itchy pale red bumps or welts on the skin. There are several types of hives including acute urticaria which last less than 6 weeks and are commonly caused by foods, medication or infections; chronic urticaria which last more than 6 weeks; and physical urticaria which are caused by something that stimulates the skin, for example cold, heat, pressure, sweating etc. In one embodiment the treatment according to the present disclosure is given for acute Hives.

Interleukin-13 receptor (IL-13R) as used herein is a type I cytokine receptor, which binds to Interleukin-13. It consists of two subunits, encoded by IL13Rα1 and IL4R, respectively. These two genes encode the proteins IL-13Rα1 and IL-4Rα. These form a dimer with IL-13 binding to the IL-13Rα1 chain and IL-4Rα stabilises this interaction. Due to the presence of the IL4R subunit, IL13R can also instigate IL-4 signalling. In both cases this occurs via activation of the Janus kinase (JAK)/Signal Transducer and Activator of Transcription (STAT) pathway, resulting in phosphorylation of STAT6. Human IL-13Rα1 has the Uniprot number P3597.

IL-13Rα2, previously called IL-13R and IL-13Rα, is another receptor which is able to bind to IL-13. However, in contrast to IL-13Rα1, this protein binds IL-13 with high affinity, but it does not bind IL-4. Human IL-13Rα2 has the Uniprot number Q14627.

In one embodiment the anti-IL-13R antibody or binding fragment thereof of the present disclosure binds to IL-13Rα1. In one embodiment, the antibody or binding fragment thereof binds only to IL-13Rα1 and does not bind to IL-13Rα2.

In one embodiment CDRH1 is an amino acid sequence GYSFTSYWIG (SEQ ID NO: 1).

In one embodiment CDRH2 is an amino acid sequence VIYPGDSYTR (SEQ ID NO: 2)

In one embodiment CDRH3 has the formula:

SEQ ID NO:3X₁Pro Asn Trp Gly X₆X₇Asp X₉
X₁ denotes Phe, Met, Gln, Leu or Val
X₆ denotes Ser or Ala
X₇ denotes Phe, Leu, Ala or Met
X₉ denotes Tyr, Gln, Lys, Arg, Trp, His, Ala, Thr,
Ser, Asn or Gly

In one embodiment the anti-IL13R antibody or binding fragment employed in the formulation of the present disclosure comprises a CDRH3 in dependently selected from SEQ ID NO: 4 to 30.

In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: or 3.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 5, 6, 7, 8, 9, 10, 11, 12, 13, 14, 15, 16, 17, 18, 19, 20, 21, 22, 23, 24, 25, 26, 27, 28, 29 or 30.

In one embodiment, the anti-IL13R antibody or binding fragment employed in the present disclosure comprises a CDRH3 having the amino acid sequence MPNWGSLDH (SEQ ID NO: 10).

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.

In one embodiment CDRL1 is an amino acid sequence RASQSISSSYLA SEQ ID NO: 31.

In one embodiment CDRL2 is an amino acid sequence GASSRAT SEQ ID NO: 32.

In one embodiment CDL3 has the formula:

SEQ ID NO:33Gln X₂X₃X₄X₅
X₂ denotes Gln, Arg, Met, Ser, Thr or Val.
X₃ denotes Tyr or Val.
X₄ denotes Glu, Ala, Gly or Ser.
X₅ denotes Thr, Ala or Ser.

In one embodiment the IL-13Rα1 antibody employed in the formulation of the present disclosure comprises a CDRL3 in dependently selected from SEQ ID NO: 34 to 47.

In one embodiment, the anti-IL13R antibody or binding fragment employed in the present disclosure comprises a CDL3 having the amino acid sequence QQYAS (SEQ ID NO: 45).

In one embodiment, the anti-IL13R antibody of the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment the IL-13R antibody employed in the present disclosure comprises a CDRL3 independently selected from a sequence comprising SEQ ID NO: 34 to 47.

In one embodiment CDRL3 comprising an amino acid sequence as set forth in SEQ ID NO: 33.

In one embodiment the VH region is independently selected from a sequence from the group comprising: SEQ ID NO: 48; SEQ ID NO: 49; SEQ ID NO: 50; SEQ ID NO: 51 and a sequence at least 95% identical to any one of the same.

### SEQ ID NO: 51

Glu Val Gln Leu Val Gln Ser Gly Ala Glu Val Lys Lys Pro Gly Glu Ser Leu Lys Ile Ser Cys Lys Gly Ser Gly Tyr Ser Phe Thr Ser Tyr Trp Ile Gly Trp Val Arg Gln Met Pro Gly Lys Gly Leu Glu Trp Met Gly Val Ile Tyr Pro Gly Asp Ser Tyr Thr Arg Tyr Ser Pro Ser Phe Gln Gly Gln Val Thr Ile Ser Ala Asp Lys Ser Ile Ser Thr Ala Tyr Leu Gln Trp Ser Ser Leu Lys Ala Ser Asp Thr Ala Met Tyr Tyr Cys Ala Arg Met Pro Asn Trp Gly Ser Leu Asp His Trp Gly Gln Gly Thr Leu Val Thr Val Ser Ser

In one embodiment the VL is independently selected from a sequence from the group comprising SEQ ID NO: 52, SEQ ID NO: 53 and SEQ ID NO: 54 and a sequence at least 95% identical to any one of the same (* K deleted in a post translational modification).

SEQ ID NO: 53
EIVLTQSPGTLSLSPGERATLSCRASQSISSSYLAWYQQKPGQAPRLLI
YGASSRATGIPDRFSGSGSGTDFTLTISRLEPEDFAVYYCQQYASFGQG
TKVEI*

In one embodiment the VH sequence is SEQ ID NO: 48 (or a sequence at least 95% identical thereto) and the VL sequence is SEQ ID NO: 52, SEQ ID NO: 53 or SEQ ID NO: 54 (or a sequence at least 95% identical to any one of the same). In one embodiment the VH sequence is SEQ ID NO: 49 (or a sequence at least 95% identical thereto) and the VL sequence is SEQ ID NO: 52, SEQ ID NO: 53 or SEQ ID NO: 54 (or a sequence at least 95% identical to any one of the same). In one embodiment the VH sequence is SEQ ID NO: 50 (or a sequence at least 95% identical thereto) and the VL sequence is SEQ ID NO: 52, SEQ ID NO: 53 or SEQ ID NO: 54 (or a sequence at least 95% identical to any one of the same). In one embodiment the VH sequence is SEQ ID NO: 51 (or a sequence at least 95% identical thereto) and the VL sequence is SEQ ID NO: 52, SEQ ID NO: 53 or SEQ ID NO: 54 (or a sequence at least 95% identical to any one of the same). In one embodiment the VL sequence is SEQ ID NO: 52 (or a sequence at least 95% identical thereto) and the VH sequence is SEQ ID NO: 48, SEQ ID NO: 49, SEQ ID NO: 50 or SEQ ID NO: 51. (or a sequence at least 95% identical to any one of the same). In one embodiment the VL sequence is SEQ ID NO: 53 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same). In one embodiment the VL sequence is SEQ ID NO: 54 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VH sequence is SEQ ID NO: 51 (or a sequence at least 95% identical thereto) and the VL sequence is SEQ ID NO: 53 ((or a sequence at least 95% identical thereto).

Variable region as employed herein refers to the region in an antibody chain comprising the CDRs and a suitable framework.

In one embodiment the heavy chain comprises a sequence independently selected from the group comprising SEQ ID NO: 55, 56, 57, 58, 59 and 60 (or a sequence at least 95% identical to any one of the same) and the light chain is independently selected from SEQ ID NO: 61 62 and 63 (or a sequence at least 95% identical to any one of the same).

In one embodiment the light chain is independently selected from a group comprising: SEQ ID NO: 61: SEQ ID NO: 62; SEQ ID NO: 63 and a sequence at least 95% identical to any one of the same.

In one embodiment the heavy chain is independently selected from SEQ ID NO: 55, 56, 57, 58, 59 and 60 (or a sequence at least 95% identical to any one of the same) and the light chain is independently selected from SEQ ID NO: 61 62 and 63 (or a sequence at least 95% identical to any one of the same).

In one embodiment the heavy chain is SEQ ID NO: 55 (or a sequence at least 95% identical thereto) and the light chain is independently selected from SEQ ID NO: 61 or 62 and 63 (or a sequence at least 95% identical to any one of the same). In one embodiment the heavy chain is SEQ ID NO: 56 (or a sequence at least 95% identical thereto) and the light chain is independently selected from SEQ ID NO: 61 or 62 and 63 (or a sequence at least 95% identical to any one of the same). In one embodiment the heavy chain is SEQ ID NO: 57 (or a sequence at least 95% identical thereto) and the light chain is independently selected from SEQ ID NO: 62 or 63 and 63 (or a sequence at least 95% identical to any one of the same). In one embodiment the heavy chain is SEQ ID NO: 58 (or a sequence at least 95% identical thereto) and the light chain is independently selected from SEQ ID NO: 61 62 and 63 (or a sequence at least 95% identical to any one of the same). In one embodiment the heavy chain is SEQ ID NO: 59 (or a sequence at least 95% identical thereto) and the light chain is independently selected from SEQ ID NO: 61 62 and 63 (or a sequence at least 95% identical to anyone of the same). In one embodiment the heavy chain is SEQ ID NO: 60 (or a sequence at least 95% identical thereto) and the light chain is independently selected from SEQ ID NO: 61 61 and 63 (or a sequence at least 95% identical to any one of the same). In one embodiment the heavy chain is SEQ ID NO: 58 or 60 (or a sequence at least 95% identical to any one of the same) and a light chain with the sequence shown in SEQ ID NO: 61 (or a sequence at least 95% identical thereto). In one embodiment the heavy chain is SEQ ID NO: 58 (or a sequence at least 95% identical to any one of the same) and a light chain with the sequence shown in SEQ ID NO: 61 (or a sequence at least 95% identical thereto). In one embodiment the heavy chain is SEQ ID NO: 60 (or a sequence at least 95% identical to any one of the same) and a light chain with the sequence shown in SEQ ID NO: 61 (or a sequence at least 95% identical thereto).

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody is eblasakimab (previously known as ASLAN004).

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

Any discussion of a protein, antibody or amino acid sequence herein will be understood to include any variants of the protein, antibody or amino acid sequence produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” (C-terminal lysine residues of encoded antibodies are often removed during the manufacturing process) and/or have part or all of a signal sequence incompletely processed and, as a consequence, remain at the terminus of the antibody. It is understood that an antibody comprising a particular amino acid sequence or binding fragment thereof may be a heterogeneous mixture of the stated or encoded sequence and/or variants of that stated or encoded sequence or binding fragment thereof.

In one embodiment the present disclosure extends to a sequence explicitly disclosed herein where the C-terminal lysine has been cleaved.

In one embodiment an antibody or binding fragment thereof, employed in a formulation of the present disclosure is humanised. Humanised (which include CDR-grafted antibodies) as employed herein refers to molecules having one or more complementarity determining regions (CDRs) from a non-human species and a framework region from a human immunoglobulin molecule (see, for example U.S. Pat. No. 5,585,089; WO91/09967). It will be appreciated that it may only be necessary to transfer the specificity determining residues of the CDRs rather than the entire CDR (see for example, Kashmiri et al 2005, Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody employed in the present invention, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

In one embodiment the anti-IL13R antibodies of the present disclosure are fully human, in particular one or more of the variable domains are fully human. Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP0438474 and EP0463151.

Thus, the presently disclosed anti-IL13R antibody may comprise one or more constant regions, such as a naturally occurring constant domain or a derivate of a naturally occurring domain.

A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or indirectly including via a coupling agent to the effector molecule. Techniques for conjugating such effector molecules to antibodies are well known in the art (see, Hellstrom et al, Controlled Drug Delivery, 2nd Ed., Robinson et al, eds., 1987, pp. 623-53; Thorpe et al, 1982, Immunol. Rev., 62:119-58 and Dubowchik et al, 1999, Pharmacology and Therapeutics, 83, 67-123). Particular chemical procedures include, for example, those described in WO93/06231, WO92/22583, WO89/00195, WO89/01476 and WO03/031581. Alternatively, where the effector molecule is a protein or polypeptide the linkage may be achieved using recombinant DNA procedures, for example as described in WO86/01533 and EP0392745.

The term effector molecule as used herein includes, for example, biologically active proteins, for example enzymes, other antibody or antibody fragments, synthetic or naturally occurring polymers, nucleic acids and fragments thereof eg DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof. Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

In patients with cancer, such as breast cancer, cancer related lymphedema (BCRL), the formulation of the present disclosure may preventlyrnphedema-associated effects, such as fibrosis, hyperkeratosis, the deposition of fibroadipose tissue, fluid accumulation, limb swelling, reduction of skin elasticity, and pain. By reducing the excess volume, said formulation may improve lymphatic and, for example limb functions.

The development of lymphedema after lymphatic injury is associated with tissue inflammation, the infiltration of CD4-positive cells and their differentiation to the type 2 helper T-cell (Th2) phenoLype. Th2 cells produce IL-4 and IL-13 that play a key role in the development of lymphedema-associated symptoms as well as other Th2-mediated diseases.

In one embodiment the formulation herein is administered in combination with another therapy. “In combination” as employed herein is intended to encompass where the anti-IL13R antibody is administered before, concurrently with another therapy or after another therapy, as the same or different formulations. Thus, combination is where the pharmacological effect of a first therapy exists at the same as the existence of a pharmacological effect of second therapy in the body and/or the two therapies are part of treatment plan designed to be employed together.

In one embodiment, a loading dose of 600 mg of the anti-IL13R antibody or binding fragment thereof is administered. In one embodiment the loading dose is administered for 1 to 3 weeks, such as 1, 2 or 3 weeks. In one embodiment the loading dose is only administered on week 0. In one embodiment the loading dose is administered on weeks 0 and 1, for example 600 mg on week 0 and 600 mg on week 1. In one embodiment the loading dose is administered on weeks 0, 1 and 2, for example 600 mg on week 0, 600 mg on week 1 and 600 mg on week 2.

Loading dose as used herein refers to a dose which is higher and/or given more frequently at the beginning of a treatment cycle. The purpose of the loading dose is to quickly “saturate” the system in vivo to provide a drug level that is therapeutic and that can be maintained by lower and/or less frequent “therapeutic” doses. In some embodiments this may be achieved locally quicker by co-locating administrations in the same part of the body. The subsequent doses given after the loading doses are referred to herein as therapeutic doses.

Therapeutic dose as employed herein refers to the amount of the anti-IL13R antibody, such as ASLAN004 that is suitable for achieving the intended therapeutic effect when employed in a suitable treatment regimen, for example ameliorates symptoms or conditions of a disease, in particular without eliciting dose limiting side effects. Suitable therapeutic doses are generally a balance between therapeutic effect and tolerable toxicity, for example where the side-effect and toxicity are tolerable given the benefit achieved by the therapy.

In one embodiment, the therapeutic dose of the anti-IL13R antibody or binding fragment thereof is 300 mg. In one embodiment, the therapeutic dose of the anti-IL13R antibody or binding fragment thereof is 300 mg dosed every 2 weeks (300 mg Q2W). In one embodiment, the therapeutic dose of the anti-IL13R antibody or binding fragment thereof is 300 mg dosed every 4 weeks (300 mg Q4W).

In one embodiment, the therapeutic dose of the anti-IL13R antibody or binding fragment thereof is 400 mg. In one embodiment, the therapeutic dose of the anti-IL13R antibody or binding fragment thereof is 400 mg dosed every 2 weeks (400 mg Q2W). In one embodiment, the therapeutic dose of the inhibitor is 400 mg dosed every 4 weeks (400 mg Q4W).

In one embodiment, the therapeutic dose of the anti-IL13R antibody or binding fragment thereof is 600 mg. In one embodiment, the therapeutic dose of the anti-IL13R antibody or binding fragment thereof is 600 mg dosed every 2 weeks (600 mg Q2W). In one embodiment, the therapeutic dose of the anti-IL13R antibody or binding fragment thereof is 600 mg dosed every 4 weeks (600 mg Q4W).

In one embodiment, the anti-IL13R antibody or binding fragment thereof is dosed at 300 mg Q2W, 400 mg Q2W, 400 mg Q4W or 600 mg Q4W. In one embodiment the anti-IL13R antibody or binding fragment thereof is dosed at 300 mg Q2W, 400 mg Q2W or 600 mg Q4W. In one embodiment, the anti-IL13R antibody or binding fragment thereof is not administered at 400 mg once every 4 weeks (400 mg Q4W).

In one embodiment, a loading dose of 600 mg of the anti-IL13R antibody or binding fragment thereof is administered at weeks 0 and 1, followed by 300 mg of the anti-IL13R antibody or binding fragment thereof dosed every two weeks. In one embodiment, a loading dose of 600 mg of the anti-IL13R antibody or binding fragment thereof is administered at weeks 0 and 1, followed by 400 mg of the anti-IL13R antibody or binding fragment thereof dosed every two weeks. In one embodiment, a loading dose of 600 mg of the anti-IL13R antibody or binding fragment thereof is administered at weeks 0, 1 and 2, followed by 400 mg of the anti-IL13R antibody or binding fragment thereof dosed every four weeks. In one embodiment, a loading dose of 600 mg of the anti-IL13R antibody or binding fragment thereof is administered at weeks 0, 1 and 2, followed by 600 mg of the anti-IL13R antibody or binding fragment thereof dosed every four weeks.

In one embodiment a formulation according to the present disclosure (including a formulation comprising same) is administered monthly, for example in a treatment cycle or as maintenance therapy.

Treatment cycle as employed herein refers to complete cycle comprising loading doses and therapeutic doses for a defined period, for example 1 month or more, 2 months or more; or 3 months or more, such as 4, 5 or 6 months. The cycle may be repeated after a break in treatment, starting again with a loading dose.

In one embodiment the treatment cycle is followed by a maintenance dose with the purpose of keeping the disease in remission.

Unit dose as used herein generally refers to a product comprising the amount of anti-IL13R antibody or binding fragment thereof of the present disclosure that is administered in a single dose including any overage. A unit dose of the presently claimed anti-IL13R antibody or antigen binding fragment thereof may refer to the marketed form of the product, such as a formulation of the anti-IL13R antibody or binding fragment thereof, wherein the product is apportioned into the amount of anti-IL13R antibody that is required for a single dose. Thus, the manufacturer is able to determine and control the exact amount of anti-13R antibody or binding fragment thereof to be included in each unit dose.

The product may be in various forms, familiar to the skilled addressee, such as vials, ampoules, infusion bags or a device (including an auto-injection device). The exact amount as employed herein refers to the amount to be administer as a dose to the patient and any overage.

In one embodiment, the unit dose or unit doses are for use according to a method of the present disclosure.

In the context of this specification “comprising” is to be interpreted as “including”.

Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section of this specification contains relevant technical information and may be used as basis for amendment.

The application claims priority from U.S. patent application No. 63/374,678 filed on 6 Sep. 2022, which is incorporated herein by reference, and may be employed to correct errors in the present specification.

Individual values or elements in the examples may be extracted and combined with generic elements of the disclosure i.e. can be used as specific basis for amendment without reference to the other features of the example.

Subject headings herein are employed to divide the document into sections and are not intended to be used to construe the meaning of the disclosure provided herein.

The present invention is further described by way of illustration only in the following examples.

### BRIEF DESCRIPTION OF FIGURES

FIG. 1A Table showing demographics of full analysis set

FIG. 1B Table showing baseline disease characteristics of full analysis set

FIG. 1C Table showing baseline disease characteristics of Evaluable for Efficacy set (EES)

FIG. 2A Table showing % change from baseline in EASI score at Day 57 for EES.

FIG. 2B Graph showing % change from baseline in EASI score at Day 57 for EES (ASLAN004 200 mg, 400 mg and 600 mg)

FIG. 2C Graph showing % change from baseline in EASI score at Day 57 for EES (ASLAN004 low dose and high doses)

FIG. 3A Table showing % change from baseline in EASI score at Day 29 for EES

FIG. 3B Graph showing % change from baseline in EASI score at Day 29 for EES (ASLAN004 200 mg, 400 mg and 600 mg)

FIG. 4A Graph showing % change in EASI score over time for EES (ASLAN004 200 mg, 400 mg and 600 mg)

FIG. 4B Graph showing % change in EASI score over time for EES (ASLAN004 low and high dose)

FIG. 5A Graph showing % change in baseline in EASI score over time for EES (Placebo)

FIG. 5B Graph showing % change in baseline in EASI score over time for EES (ASLAN004 200 mg)

FIG. 5C Graph showing % change in baseline in EASI score over time for EES (ASLAN004 400 mg)

FIG. 5D Graph showing % change in baseline in EASI score over time for EES (ASLAN004 600 mg)

FIG. 6A Table showing Day 57 sensitivity analysis in the modified intention to treat (mITT)

FIG. 6B Graph showing Day 57 sensitivity analysis in the mITT (ASLAN004 200 mg, 400 mg and 600 mg)

FIG. 6C Graph showing Day 57 sensitivity analysis in the mITT (ASLAN004 low and high dose)

FIG. 7A Summary table showing EASI 50, EASI 75, EASI 90 at Day 57 for EES

FIG. 7B Graph showing EASI 50 at Day 57 for EES (ASLAN004 200 mg, 400 mg and 600 mg)

FIG. 7C Graph showing EASI 50 at Day 57 for EES (ASLAN004 low and high dose)

FIG. 7D Graph showing EASI 75 at Day 57 for EES (ASLAN004 200 mg, 400 mg and 600 mg)

FIG. 7E Graph showing EASI 75 at Day 57 for EES (ASLAN004 low and high dose)

FIG. 7F Graph showing EASI 90 at Day 57 for EES (ASLAN004 200 mg, 400 mg and 600 mg)

FIG. 7G Graph showing EASI 90 at Day 57 for EES (ASLAN004 low and high dose)

FIG. 8A Graph showing proportion of patients achieving EASI 50

FIG. 8B Graph showing proportion of patients achieving EASI 75

FIG. 8C Graph showing proportion of patients achieving EASI 90

FIG. 9 Summary table showing proportion of patients achieving EAS I50, 75, 90 for EES and mITT

FIG. 10A Summary table showing proportion of patients with IGA score of 0 or 1 at Day 57 for ESS

FIG. 10B Graph showing proportion of patients with IGA score of 0 or 1 at Day 57 for ESS

FIG. 10C Graph showing proportion of patients with IGA score of 0 or 1 over time for ESS

FIG. 11 Table showing baseline TARC and IgE levels of patients

FIG. 12A Graph showing average % change from baseline TARC (ASLAN004 200 mg and 400 mg)

FIG. 12B Graph showing average % change from baseline TARC (ASLAN004 400 mg and placebo)

FIG. 12C Graph showing % change from baseline TARC for individual patients (ASLAN004 400 mg)

FIG. 13A Graph showing IgE % change from baseline for ASLAN004 200 mg and 400 mg

FIG. 13B Graph showing average IgE % change from baseline for ASLAN004 200 mg and 400 mg

FIG. 13C Graph showing IgE % change from baseline for individual patients for placebo

FIG. 13D Graph showing IgE % change from baseline for individual patients for ASLAN004 200 mg

FIG. 13E Graph showing IgE % change from baseline for individual patients for ASLAN004 400 mg

FIG. 14 Graph showing comparison between ASLAN004 exposure, EASI, TARC and IgE over time for patients receiving ASLAN004 400 mg

FIG. 15 Table showing comparison between ASLAN004 and Duplilumab

FIG. 16 Flow chart showing number of subjects in each test group

FIG. 17 Table showing baseline demographics and disease characteristics of Intention-to-treat (ITT), modified Intention-to-treat (mITT) and Excluded site groups

FIG. 18 Table showing adverse events (AEs) for mITT vs Excluded site groups

FIG. 19A Graph showing improvement in (median) worst itch over time for mITT group

FIG. 19B Graph showing improvement in (median) worst itch at week 8 for mITT group

FIG. 20A Graph showing improvement in (median) average itch over time for mITT group

FIG. 20B Graph showing improvement in (median) average itch at week 8 for mITT group

FIG. 21 Graph showing improvement in POEM score change from baseline (CFBL) over time

FIG. 22 Graph showing 2-point improvement in sleep disturbance (SD) score at week 8 for miTT group

FIG. 23 Table showing baseline characteristics of Intention-to-treat (ITT) patients from TREK-AD study. ^{a}=Ebla 400 mg Q4W (n=59); Ebla 600 mg Q4W (n=57); Ebla 300 mg Q2W (n=58); Ebla 400 mg Q2W (n=55); Placebo Q2W (n=56); All Ebla (n=229) ^{b}=Ebla 400 mg Q4W (n=24); Ebla 600 mg Q4W (n=21); Ebla 300 mg Q2W (n=20); Ebla 400 mg Q2W (n=19); Placebo Q2W (n=23); All Ebla (n=84) ^{c}=Ebla 400 mg Q4W (n=24); Ebla 600 mg Q4W (n=20); Ebla 300 mg Q2W (n=20); Ebla 400 mg Q2W (n=19); Placebo Q2W (n=23); All Ebla (n=83) BSA, body surface area; DLQI, Dermatology Life Quality Index; EASI, Eczema Area and Severity Index; IGA, investigator global assessment (5-point scale); NRS, numeric rating scale; POEM, patient oriented eczema measure; Q2W, every 2 weeks; Q4W, every 4 weeks; SD, standard deviation

FIG. 24 Graph showing % mean change from baseline for sleep deprivation component of SCORAD for ITT patients from TREK-AD study over 16 weeks

FIG. 25A Graph showing proportion of ITT patients with baseline SD NRS achieving a sleep disturbance numerical rating scale (SD NRS) change of ≥4 points over the course of the 16 week study

FIG. 25B Graph showing proportion of ITT patients with baseline SD NRS achieving a sleep disturbance numerical rating scale (SD NRS) change of ≥4 points at week 16

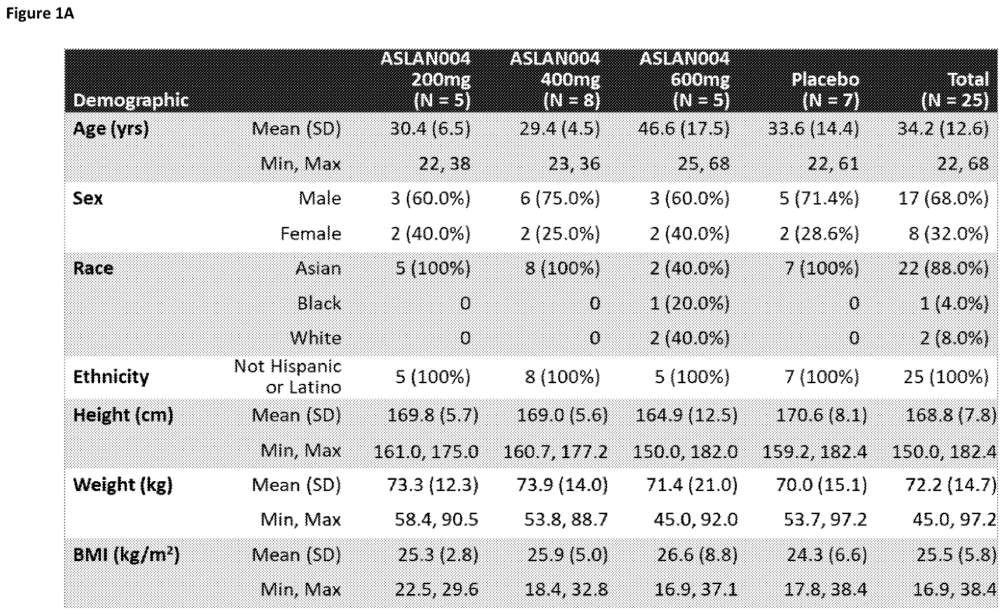

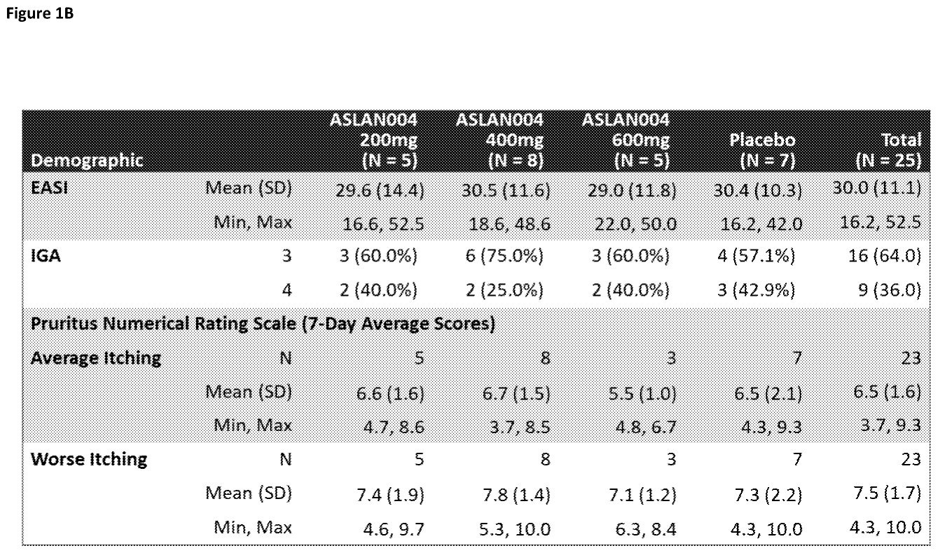

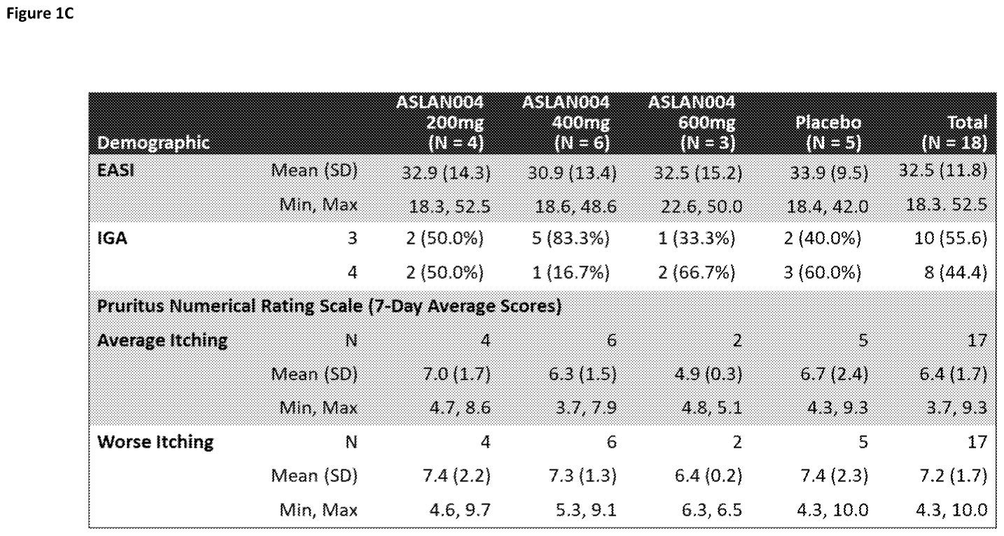

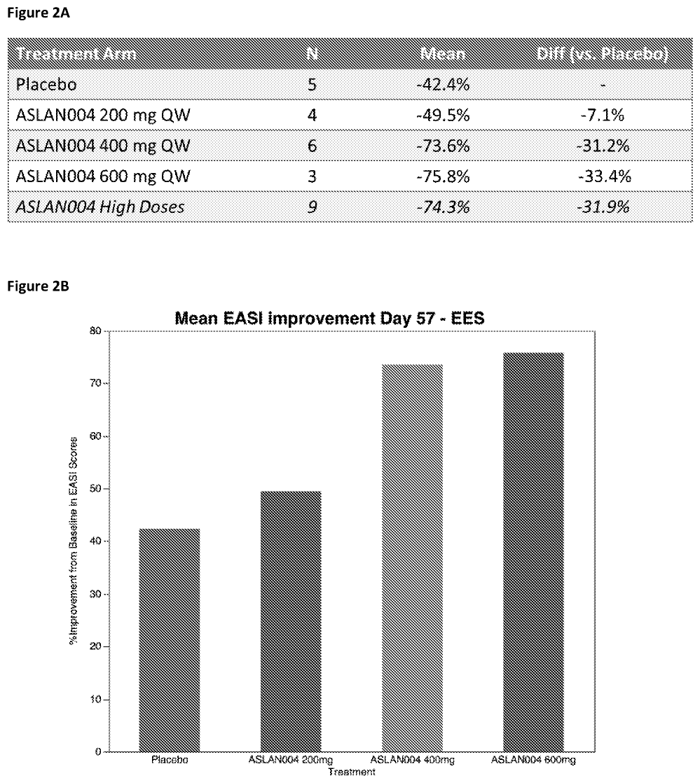

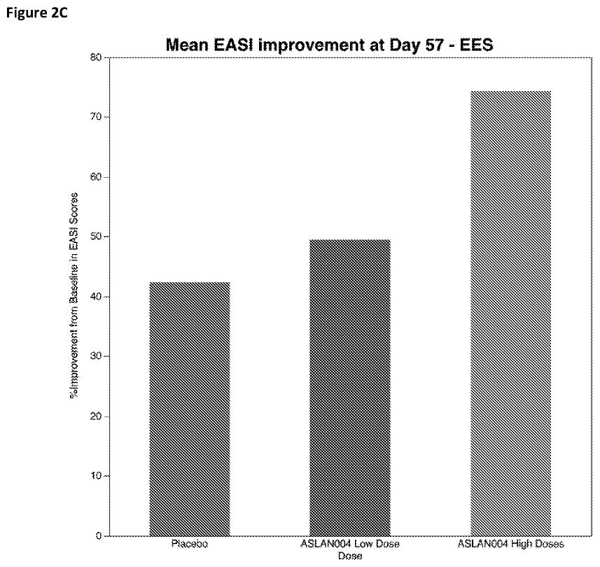

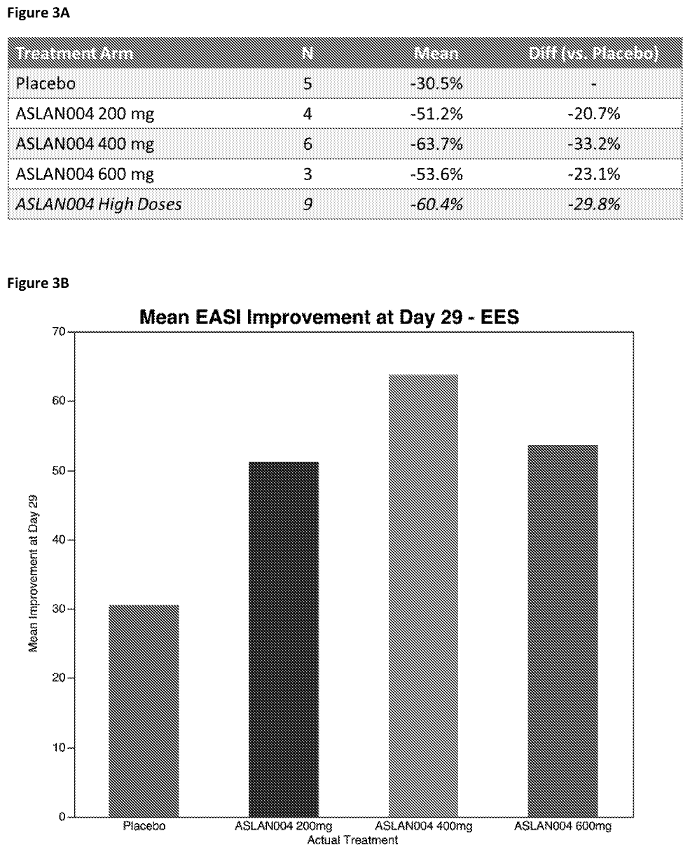

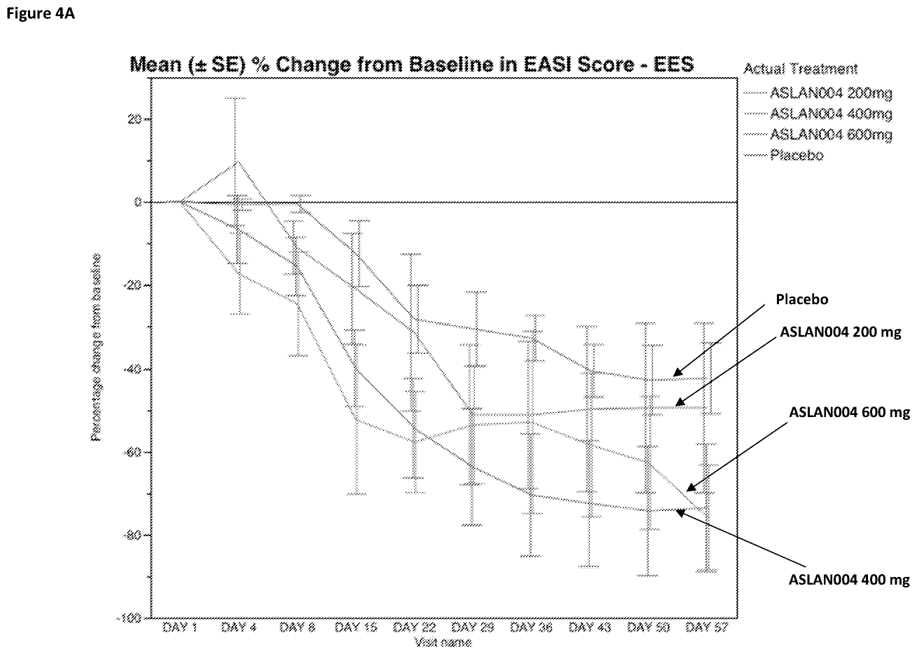

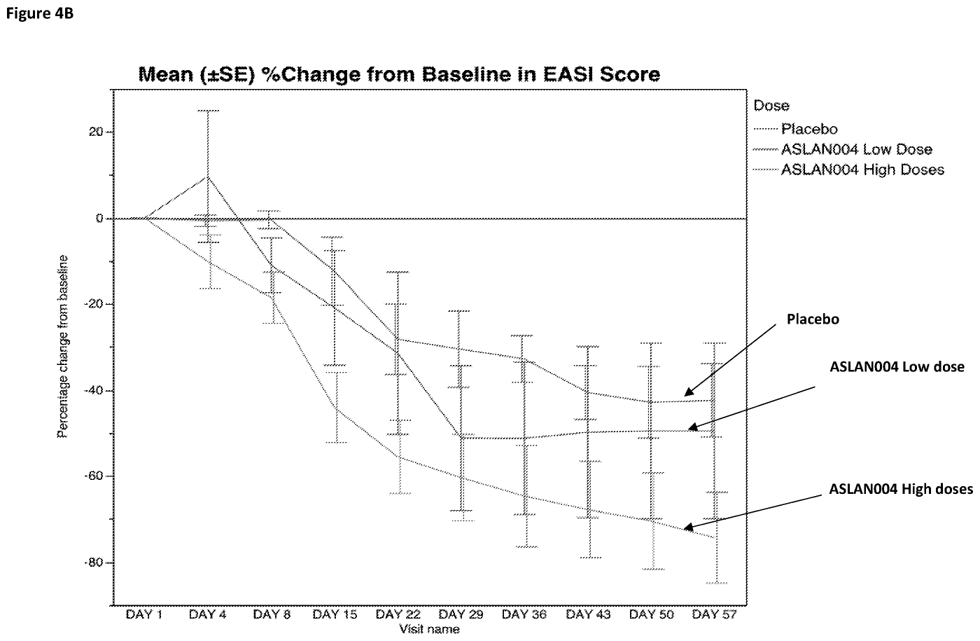

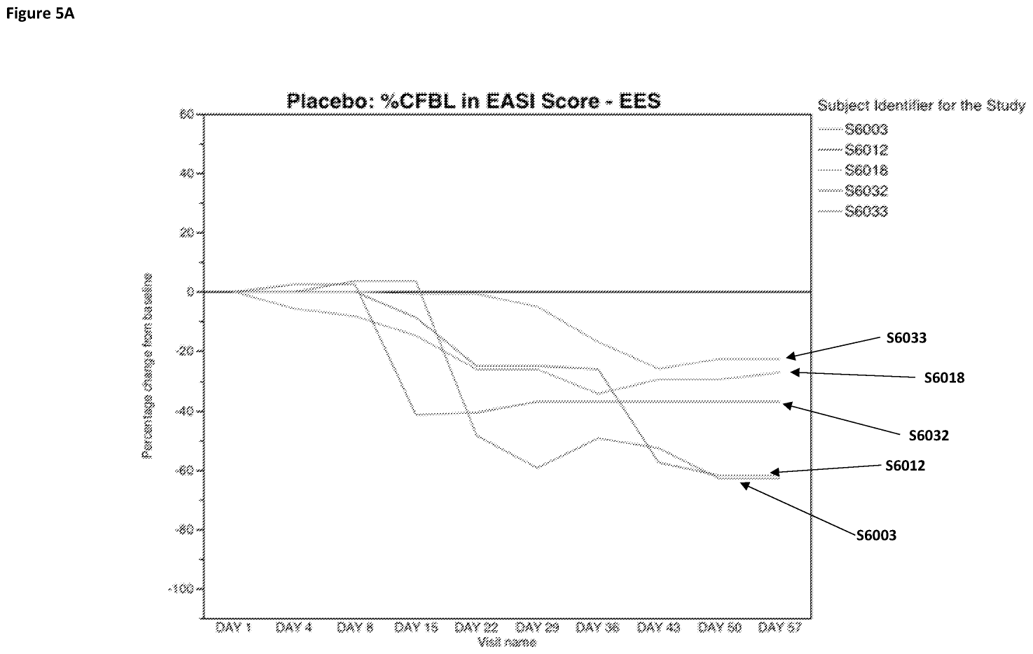

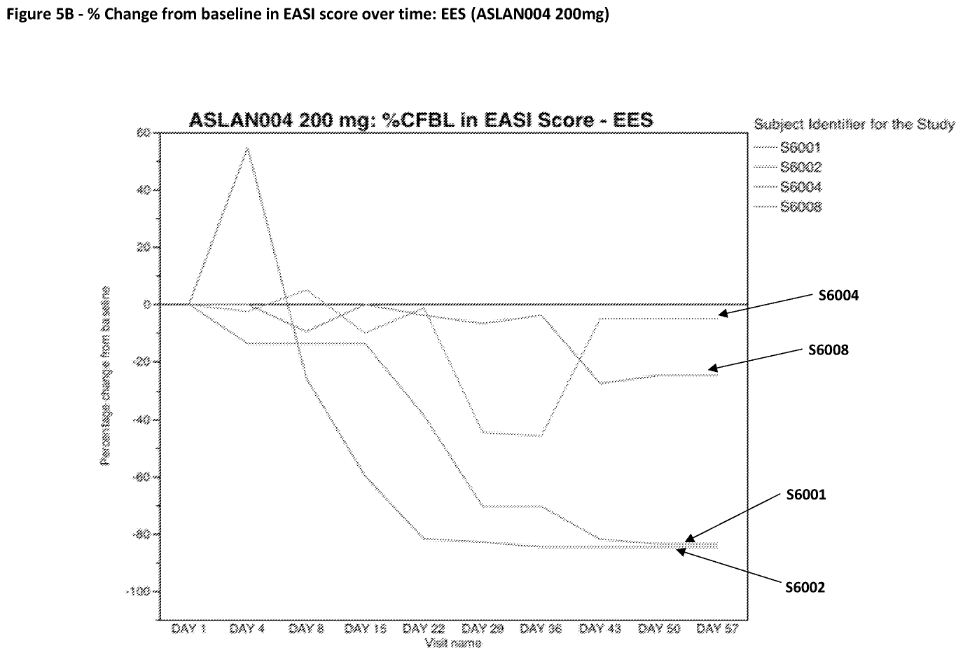

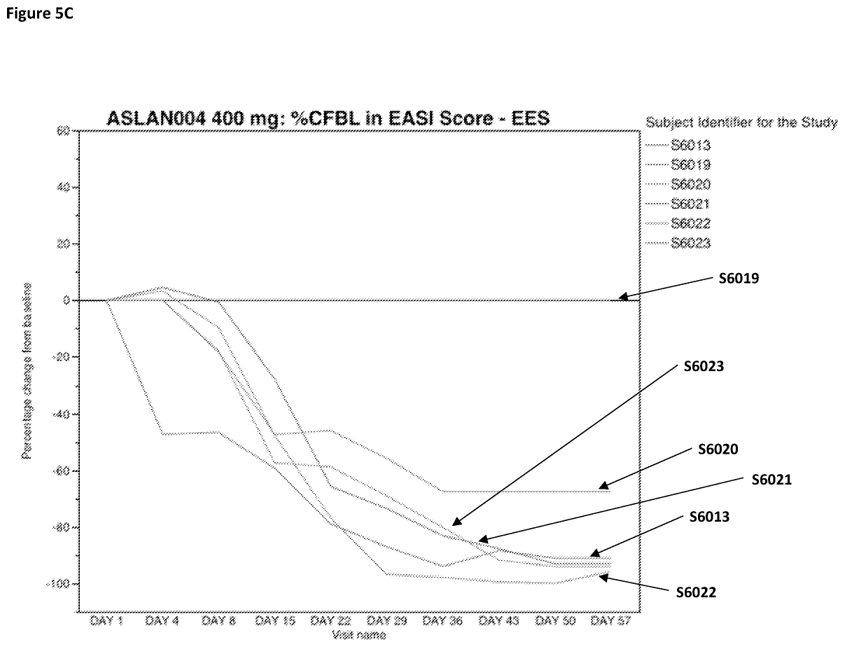

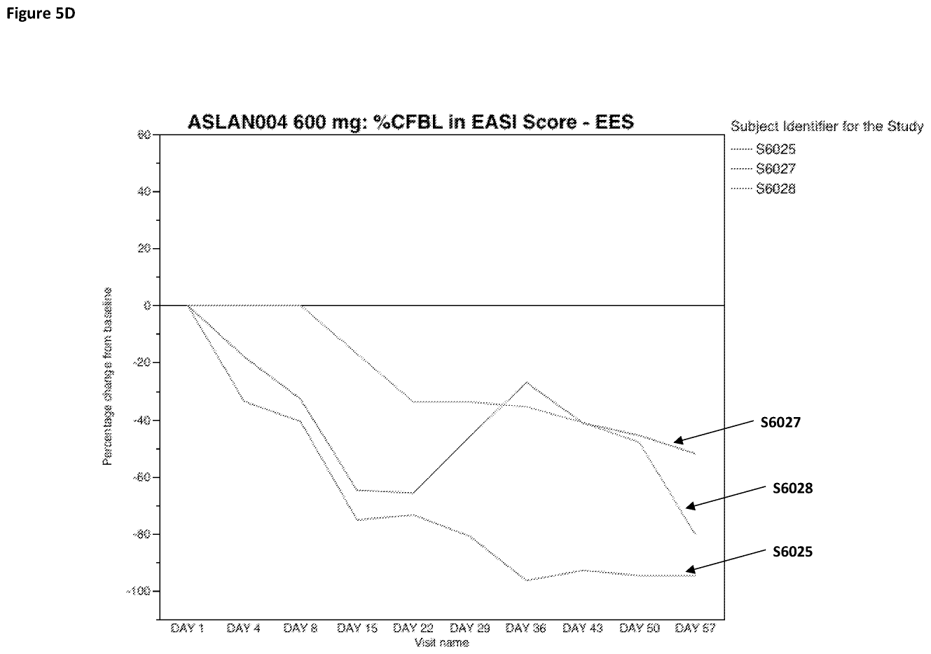

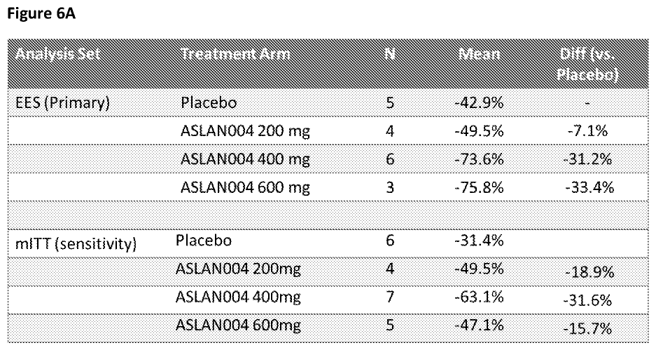

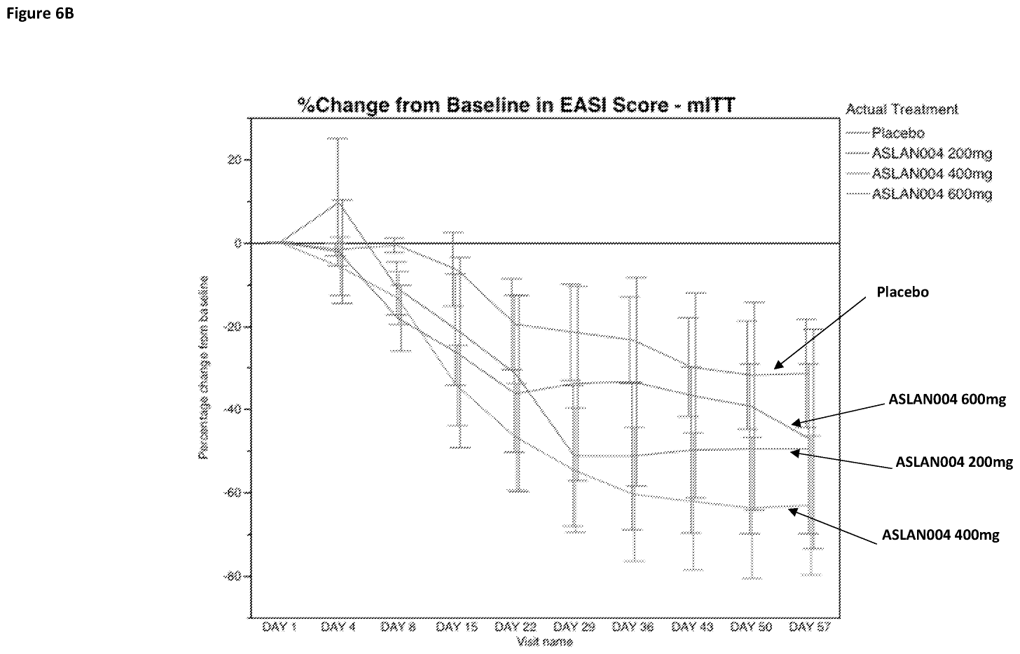

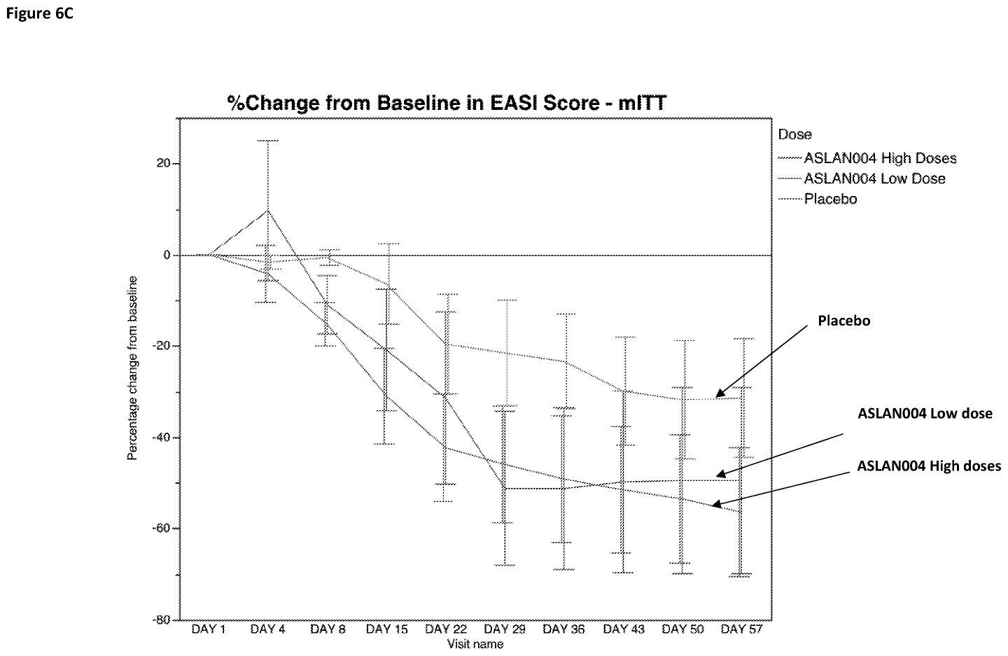

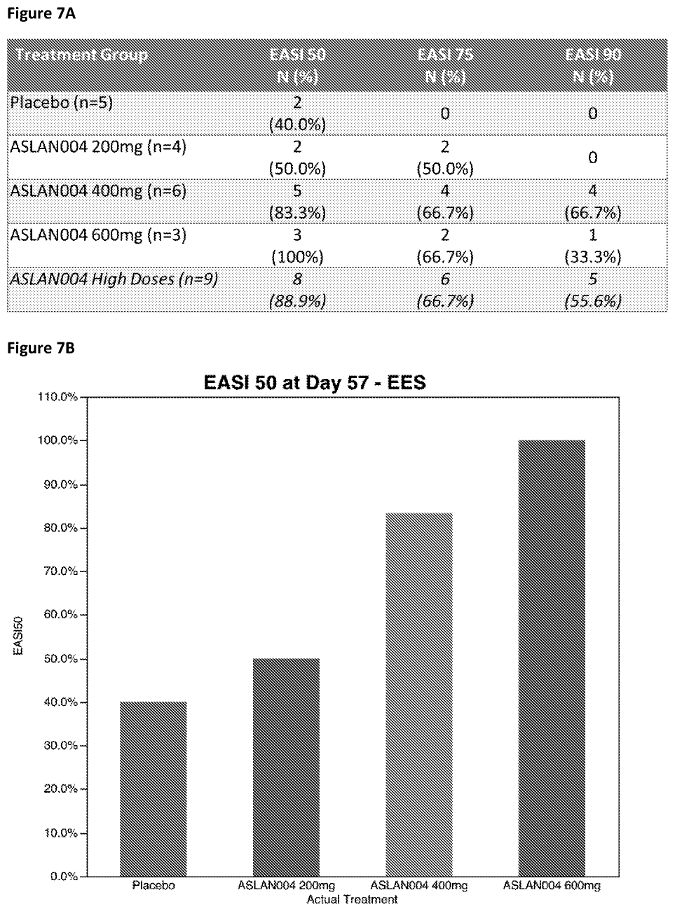

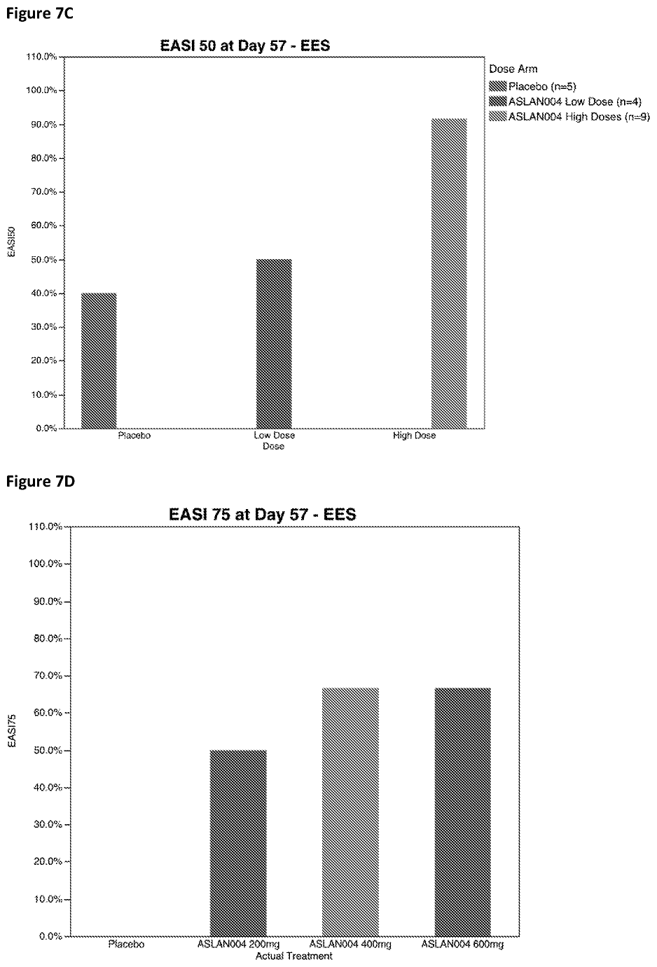

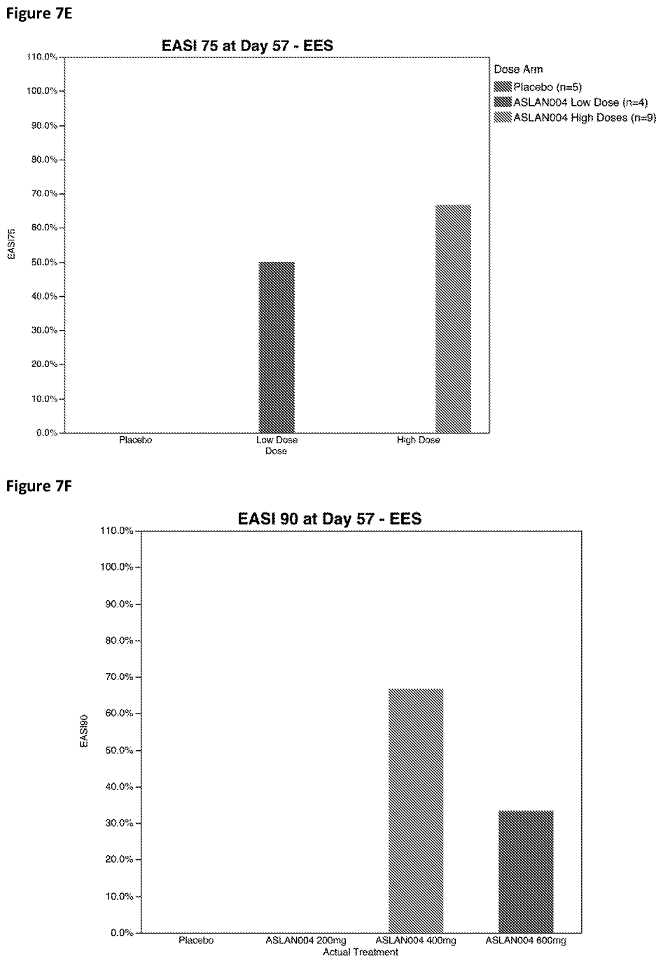

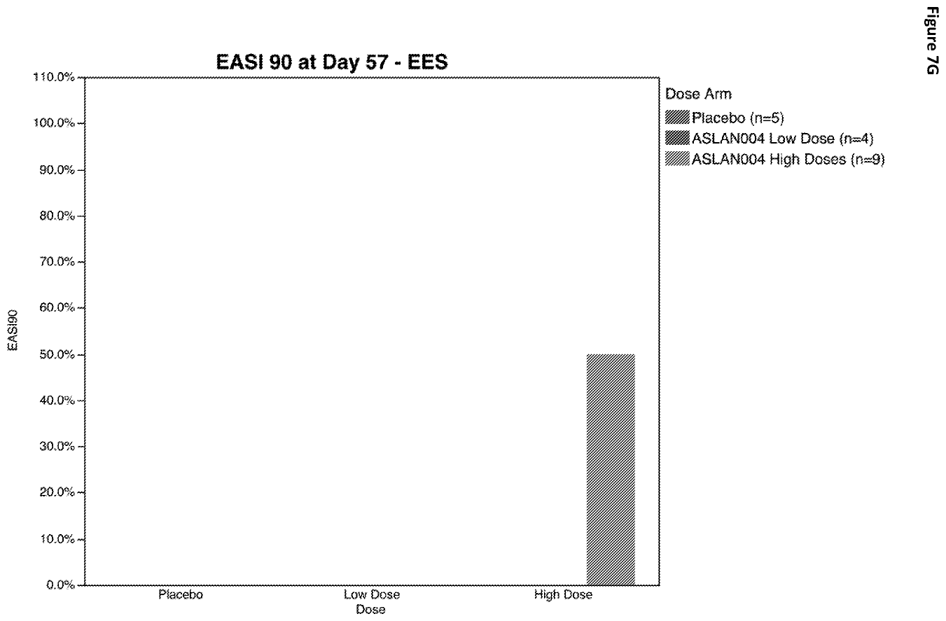

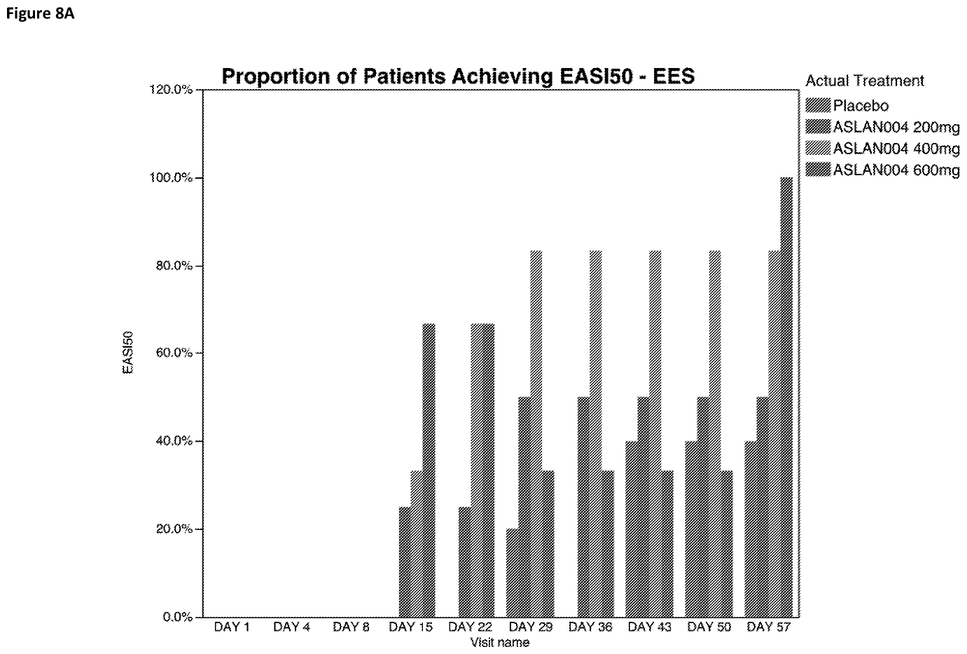

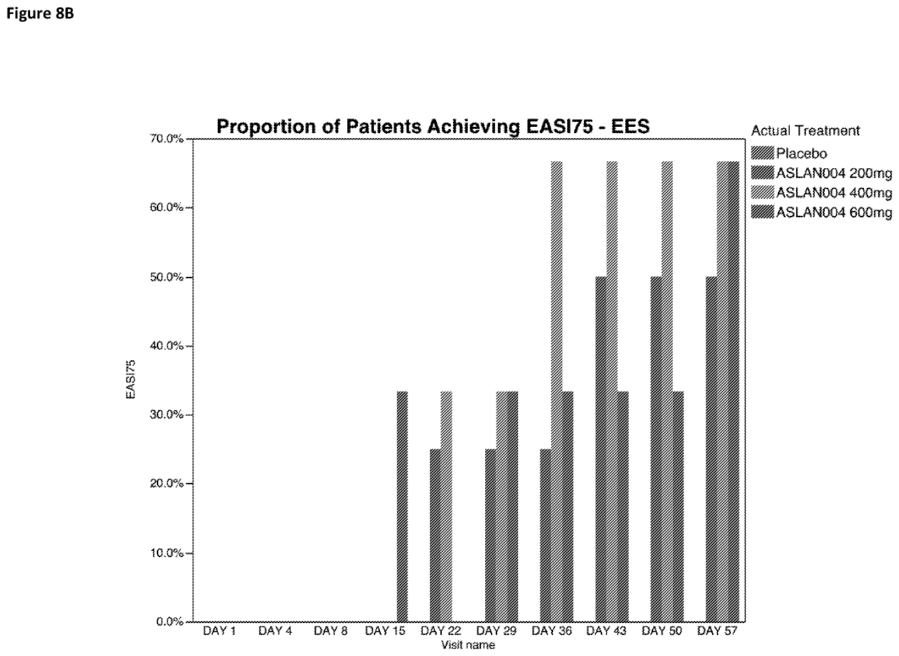

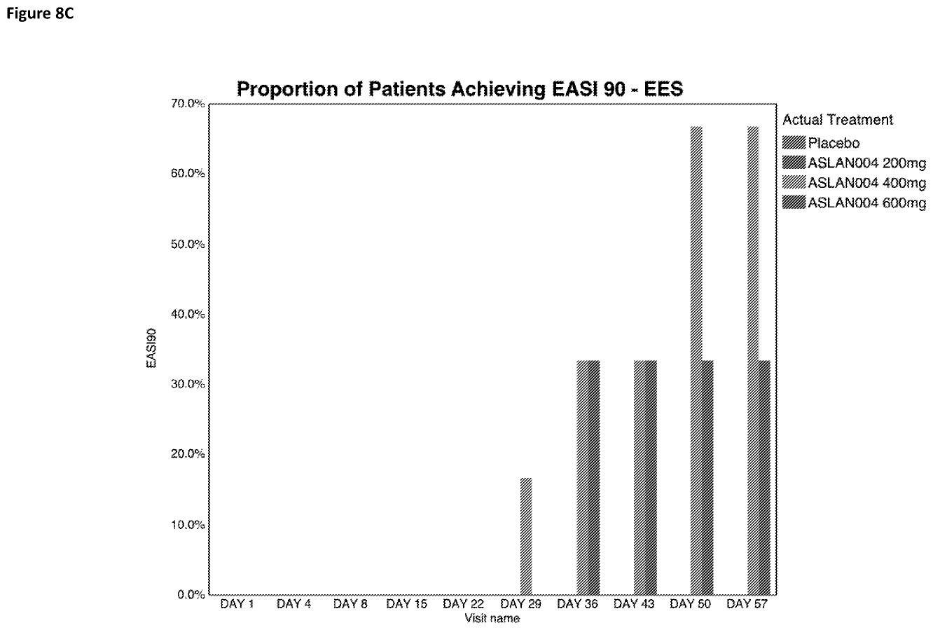

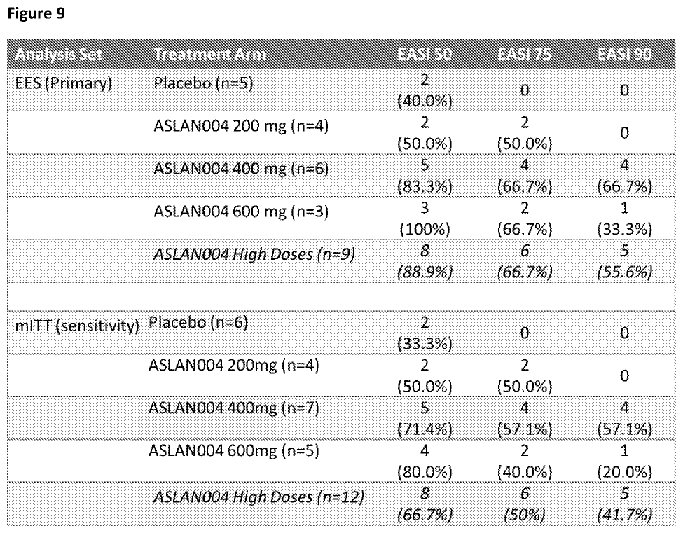

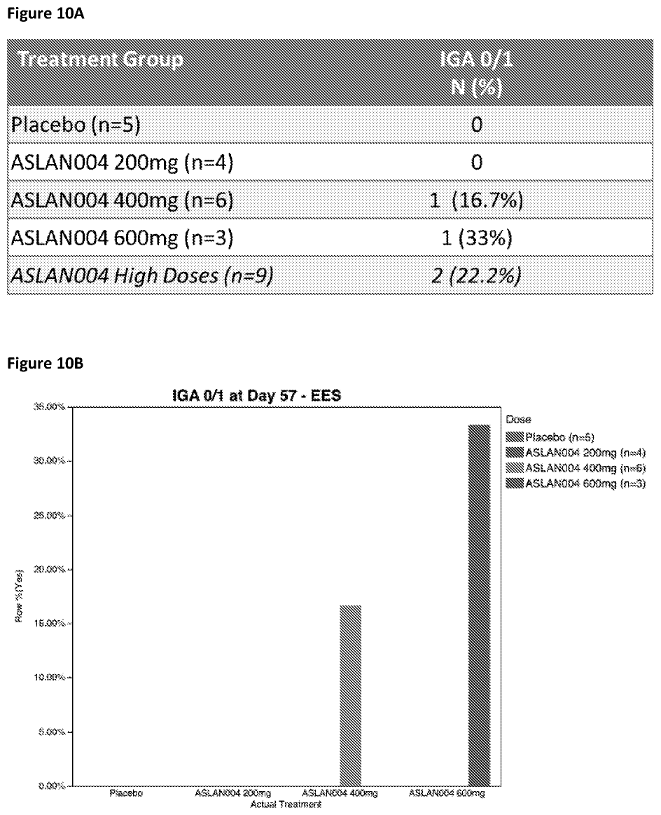

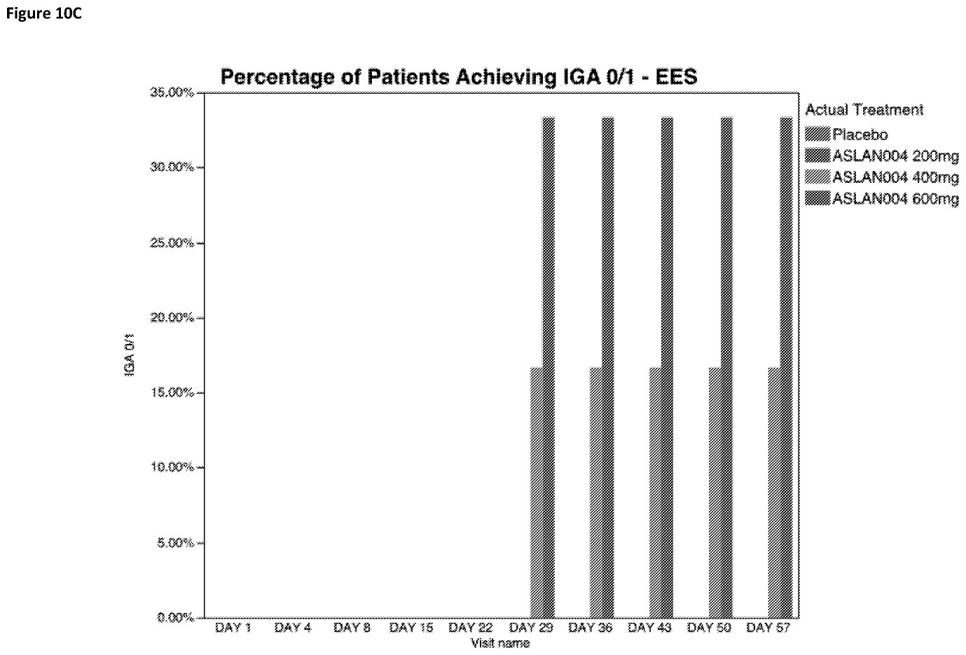

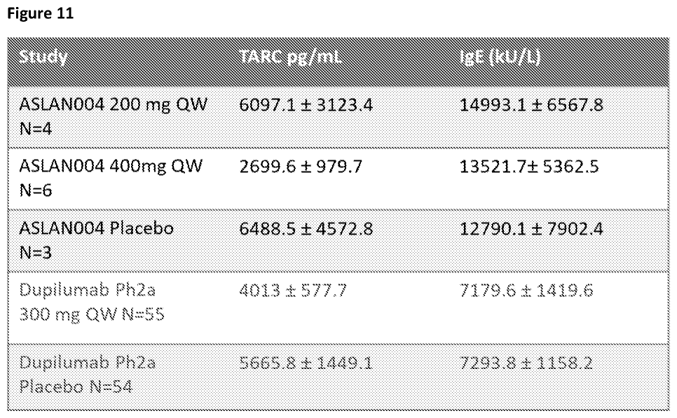

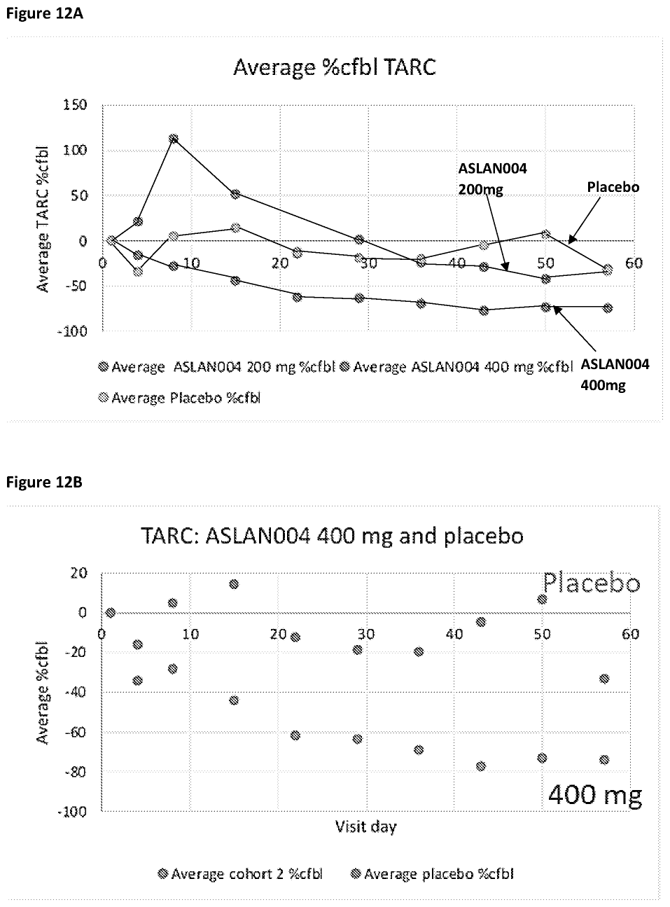

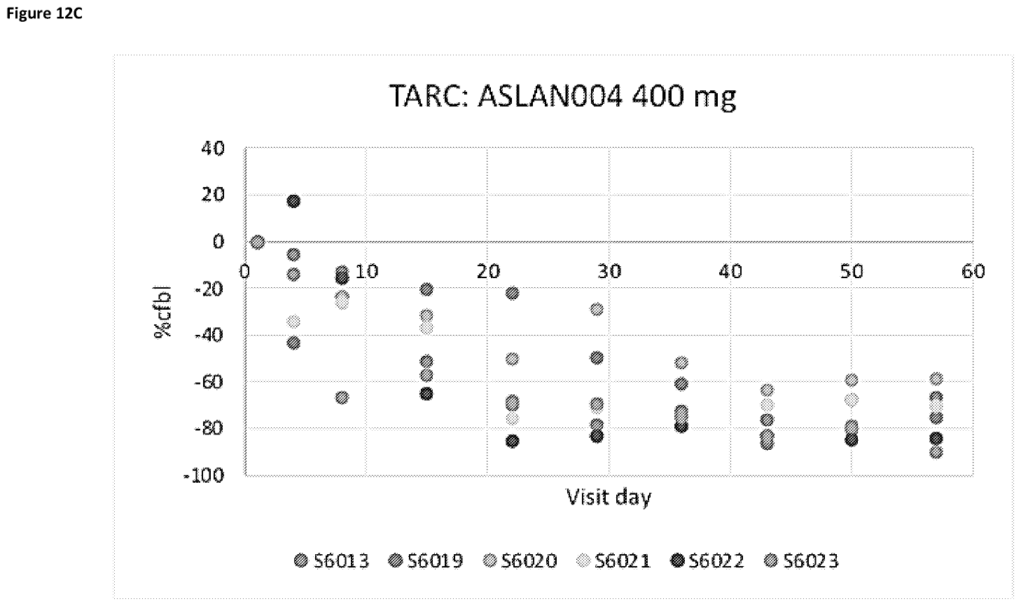

### EXAMPLES

### Example 1 Study Protocol (Initial MAD Escalation)

Patients enrolled in ascending dose cohorts of ASLAN004 (SEQ ID NO: 51, 53 and 59 herein): 200 mg, 400 mg, 600 mg. Initially the doses were given QW. Within each cohort, patients were randomized in a 3:1 ratio of ASLAN004:Placebo

TABLE 1
Baseline TARC and IgE

| Study | TARC pg/mL | IgE (kU/L) |
|---|---|---|
| ASLAN004 200 mg QW | 6097.1 ± 3123.4 | 14993.1 ± 6567.8 |
| N = 4 | | |
| ASLAN004 400 mg QW | 2699.6 ± 979.7 | 13521.7 ± 5362.5 |
| N = 6 | | |
| ASLAN004 Placebo | 6488.5 ± 4572.8 | 12790.1 ± 7902.4 |
| N = 3 | | |
| Dupilumab Ph2a | 4013 ± 577.7 | 7179.6 ± 1419.6 |
| 300 mg QW N = 55 | | |
| Dupilumab Ph2a | 5665.8 ± 1449.1 | 7293.8 ± 1158.2 |
| Placebo N = 54 | | |

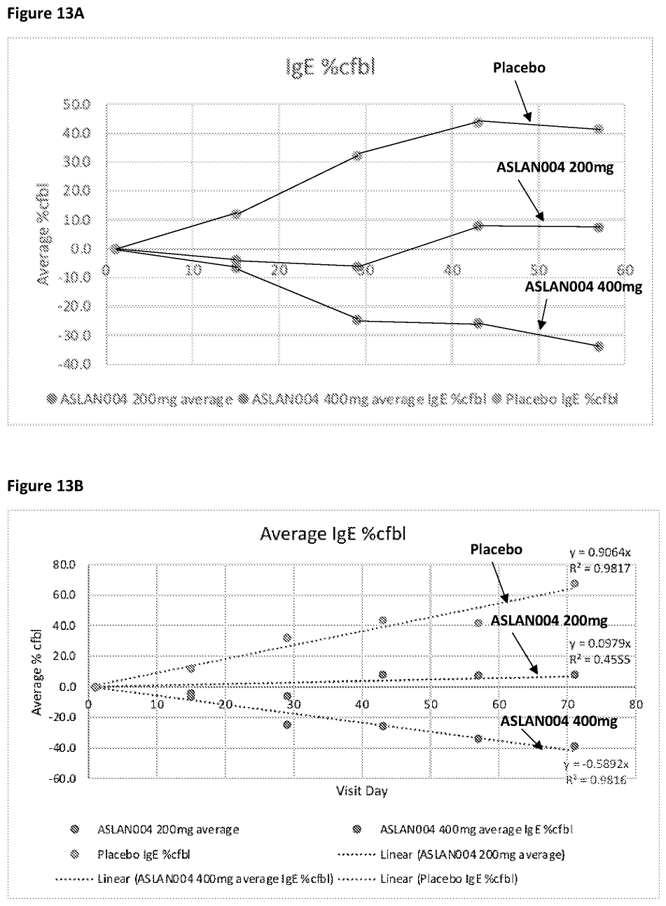

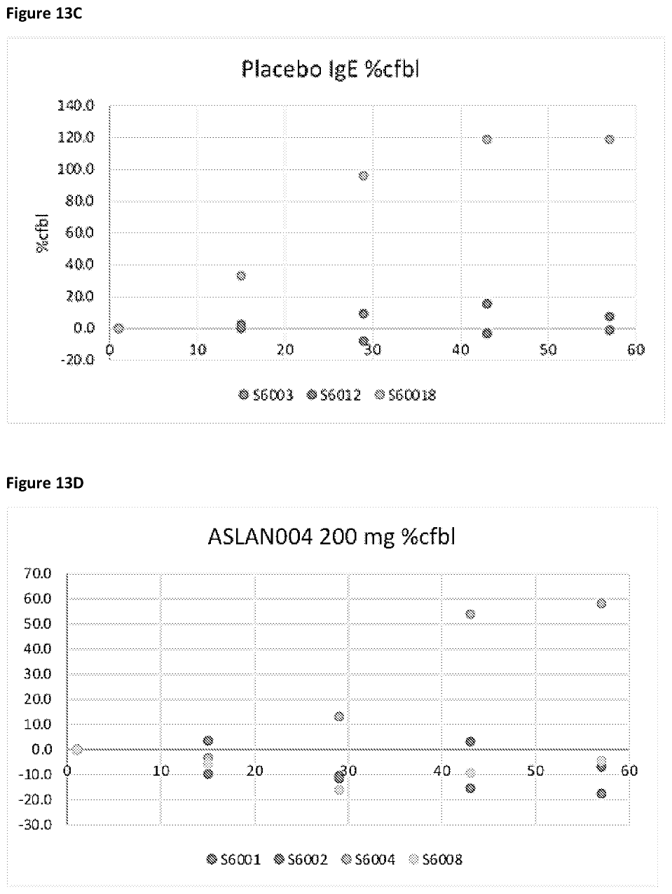

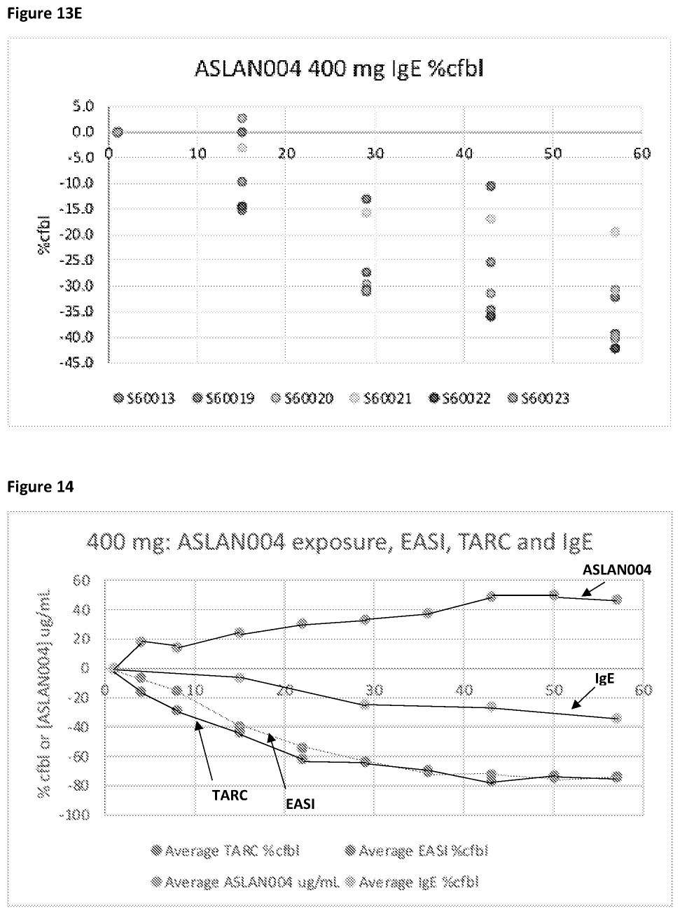

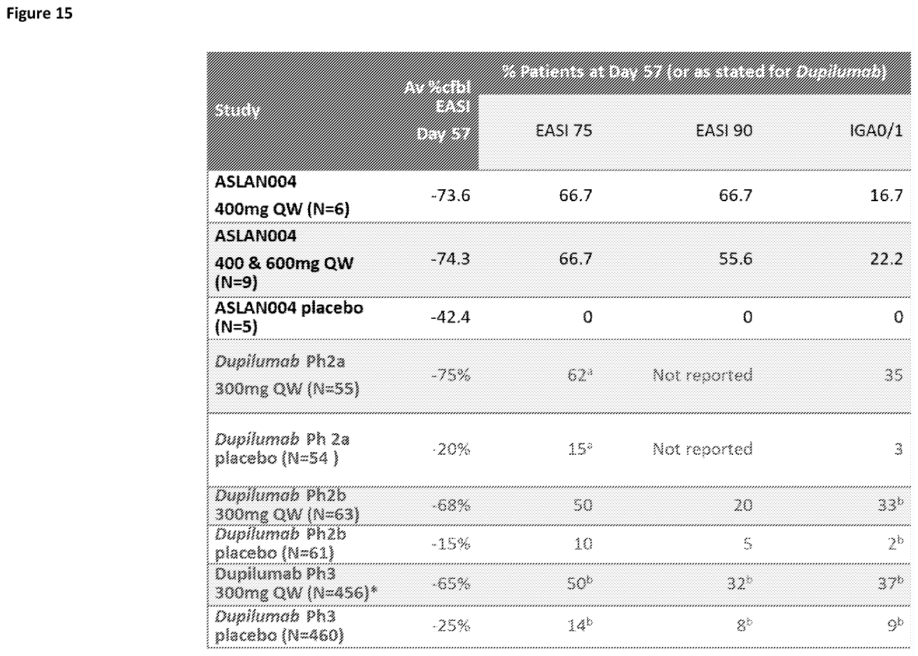

FIG. 13A shows the % change from baseline at day 15, 29, 43 and 57 or all patients receiving treatment FIG. 13B shows the average % change from baseline over the same period.

FIG. 13C to E shows the data for the individual patients.

TABLE 2
Shows a reduction and −negative number in IgE levels

| | Placebo | Drug |
|---|---|---|
| Study | % cfbl | % cfbl |
| ASLAN004 | 41.6 | −34 |
| 400 mg QW | | |
| Dupilumab | 15 | −35 |
| 300 mg QW Ph2a | | |
| Dupilumab | 10 | −15 |
| 300 mg QW Ph2b/Ph3 | 15 | −30 |

It can be seen that in highly allergic population of patients (IgE 41.6% cfbl) ASLAN004 reduced the IgE levels by −34%. In real terms this is a more significant reduction than the −15 to −30% in a population with lower allergic response in dupilumab.

### Example 2

In the 32 patients that completed at least 29 days of dosing across all sites, defined in the protocol as the efficacy evaluable data set, the average reduction from baseline in EASI at 8 weeks was 73% (n=19) compared to 44% (n=13) for patients on placebo (p=0.007¹).

The proportion of patients with adverse events and treatment-related adverse events were similar across treatment and placebo arms. There were no incidences of conjunctivitis in the expansion cohort.

TABLE 3
results from revised intention to treat (RITT) and intention to treat (ITT) groups

| | RITT (n = 29) | | | ITT (n = 38) | | |
|---|---|---|---|---|---|---|
| | 600 mg | Placebo | p- | 600 mg | Placebo | p- |
| Endpoint | (n = 16) | (n = 13) | value¹ | (n = 22) | (n = 16) | value¹ |
| Mean % change from | −64.9 | −27.2 | 0.021 | −61.3 | −31.9 | 0.023 |
| baseline in EASI | | | | | | |
| EASI-50 (%) | 81.3 | 30.8 | 0.008 | 77.3 | 37.5 | 0.016 |
| EASI-75 (%) | 68.8 | 15.4 | 0.005 | 50.0 | 12.5 | 0.018 |
| EASI-90 (%) | 37.5 | 15.4 | 0.183 | 27.3 | 12.5 | 0.245 |
| IGA 0/1 (%) | 43.8 | 15.4 | 0.107 | 31.8 | 18.8 | 0.301 |
| Mean % change from | −38.6 | −15.3 | 0.051 | −37.1 | −15.7 | 0.032 |
| baseline in peak | | | | | | |
| pruritis Numerical | | | | | | |
| Rating Scale | | | | | | |
| Mean change from | −9.8 | −2.5 | 0.007 | −9.0 | −3.5 | 0.014 |
| baseline in POEM | | | | | | |

¹One-sided p-value

ASLAN004 achieved a statistically significant improvement (p<0.025) versus placebo in the primary efficacy endpoint of percent change from baseline in the Eczema Area Severity Index (EASI), and also showed significant improvements (p<0.05) in other key efficacy endpoints: EASI-50, EASI-75, peak pruritis and the Patient-Oriented Eczema Measure (POEM).

Following discussions with the Data Monitoring Committee prior to unblinding, a Revised ITT population (RITT, n=29) was defined to exclude one study site at which all patients enrolled in the study appeared atypical of moderate-to-severe AD patients based on biomarkers, such as TARC, and patient medical history. In the RITT population, which is more comparable to other published studies in moderate-to-severe AD, ASLAN004 also achieved a statistically significant improvement (p<0.025) versus placebo in percent change from baseline in EASI and showed a greater improvement over placebo in the key efficacy endpoints versus the ITT population.

### Example 3

The objective of this study was to evaluate the effects of eblasakimab on itch and sleep scores in AD.

### Methods

Three patient cohorts were randomized to receive either 200, 400 or 600 mg eblasakimab or placebo subcutaneously once weekly for 8 weeks in a multiple ascending dose study design.

Adult patients were included with chronic AD present for ≥3 years before screening, and the following AD parameters at screening and baseline: eczema area and severity index (EASI)≥16, Investigator's Global Assessment (IGA) score ≥3 (scale of 0 to 4), and ≥10% body surface area (BSA) of AD involvement Rescue medication (moisturizer with active ingredient, topical corticosteroids, topical calcineurin inhibitors) was not allowed; LOCF was used for participants who used rescue medication.

Patient reported outcomes were measured, including pruritus numeric rating scale (P-NRS) for both worst and average itch and Patient-Oriented Eczema Measure (POEM), which includes a single item sleep loss component.

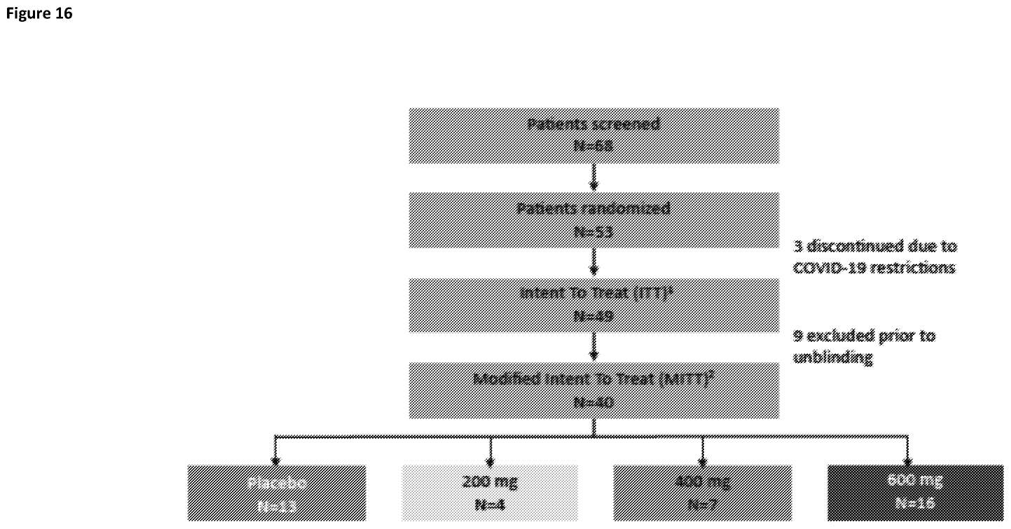

Inferential statistical analysis was performed for 600 mg vs. placebo groups at week 8 only; results for 200 and 400 mg groups were descriptively described due to small sample size. Efficacy analysis in the Phase 1b study used a modified Intent to Treat (mITT) population in which 9 study patients from one site were excluded (excluded site group) from the ITT analysis prior to unblinding as the participants did not have disease characteristics consistent with moderate to severe AD (FIG. 16).

### Results

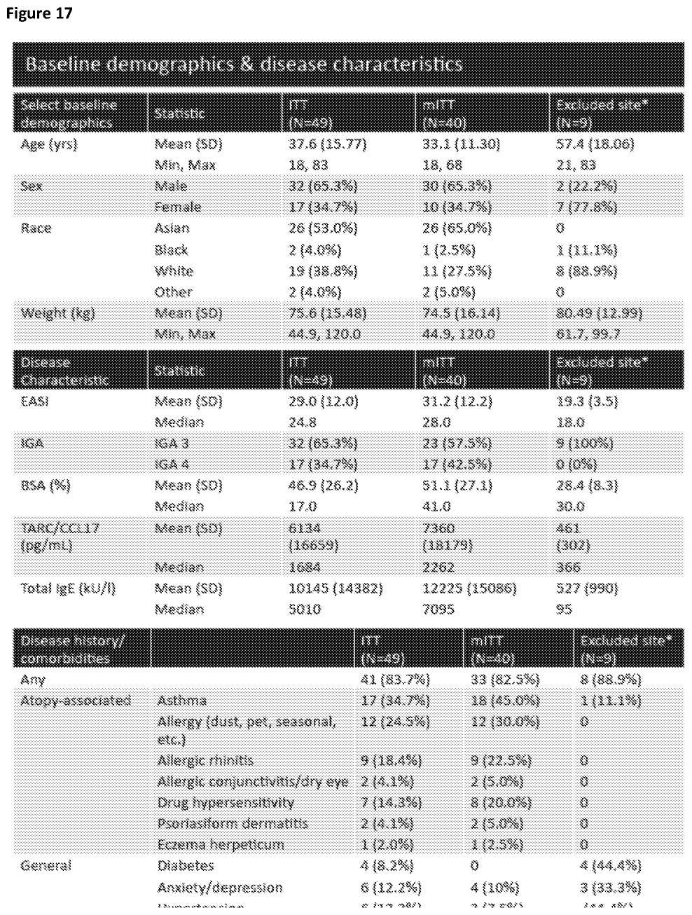

The Excluded site set was markedly different from the mITT set at baseline with substantially lower serum TARC/CCL17 (7,350 pg/mL and 461 μg/mL, respectively), serum IgE (12,225 kU/I vs 527 kU/I), and EASI scores (mean 31.2 vs 19.3) showing lower extent and severity of disease. Other notable differences included older age, and lower IGA, BSA and POEM scores. Participants in this site had no atopic disease history but reported other comorbidities including diabetes and hypertension (FIG. 17).

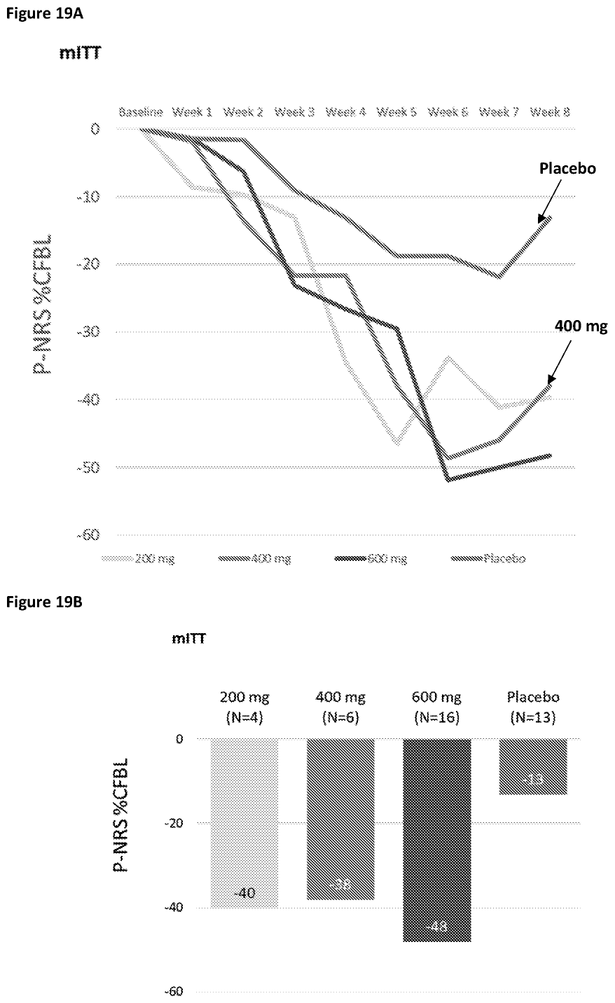

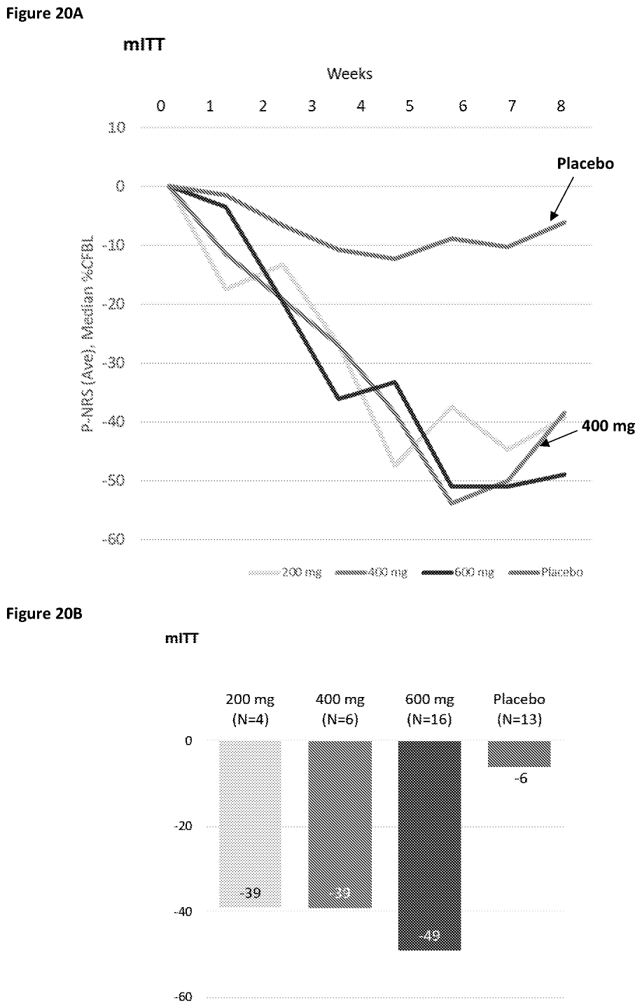

Improvements in percent change from baseline (% CFBL) in P-NRS for median worst itch were apparent over time and at week 8 (FIGS. 19A & 19B) and also for median average itch (FIGS. 20A & 20B) with eblasakimab treatment compared with placebo (worst itch: −48% vs. −13%; average itch: −49% vs. −6%, eblasakimab 600 mg vs. placebo, respectively) in the mITT analysis set Participants receiving eblasakimab 600 mg in the Excluded site* exhibited improvement in average itch but not worst itch vs. placebo.

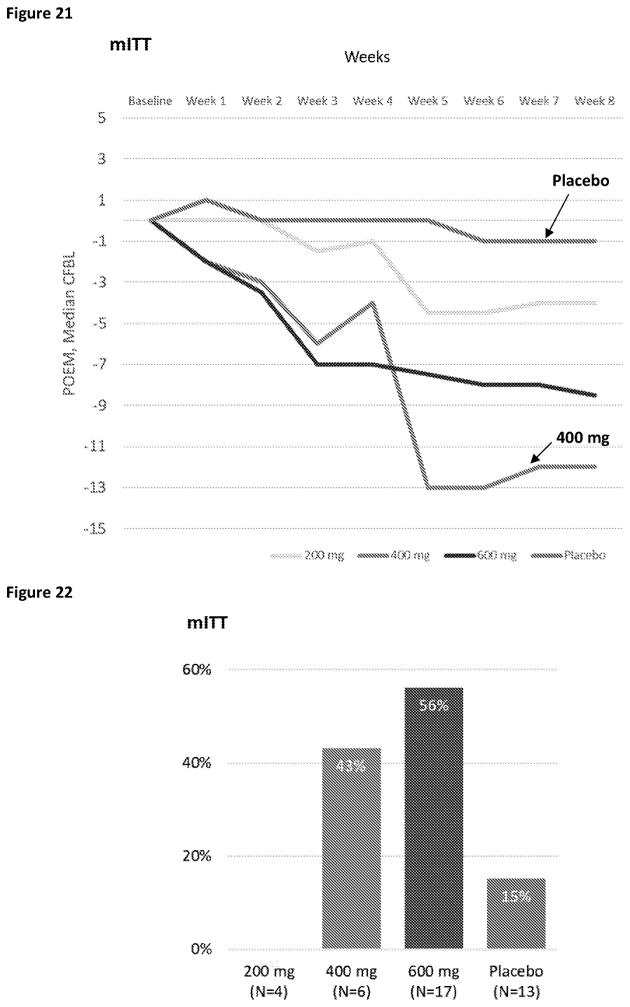

Improvements in POEM score were apparent over time with eblasakimab treatment compared with placebo in the mITT set, with the 400 and 600 mg doses producing a greater magnitude of response than the 200 mg dose (FIG. 21). At week 8, median POEM CFBL for 400 mg and 600 mg eblasakimab was −12 and −9 respectively, vs. −1 for placebo in the mITT set No improvement vs. placebo was observed in the Excluded site group.

A 4-point improvement in POEM score was observed at week 8 for eblasakimab 600 mg vs. placebo in the mITT but not the Excluded site analysis sets (81% vs. 23%; 50% vs. 100%, respectively). There was a greater improvement in POEM sleep scores with eblasakimab vs. placebo (FIG. 22). A 2-point improvement (mean) in sleep loss (POEM item) was observed at week 8 for 400 mg and 600 mg eblasakimab (43% and 56% vs. 15% for placebo) in the mITT analysis set, and 600 mg eblasakimab in the Excluded site (33% vs. 0% for placebo).

Importantly, these are improvements for patients with sleep disturbance scores of 3 or 4 at the baseline. The majority of patients (75% [12/16]) in the 600 mg treatment group reported ≥5 nights of sleep disturbance at baseline vs. placebo (54% [7/13]). More (63% [10/16]) of eblasakimab-treated patients reported ‘no days’ or ‘1-2 days’ of sleep disturbance at week 8 vs. placebo (38% [5/13])

Rescue medication use was low, but higher in the placebo group (data not shown).

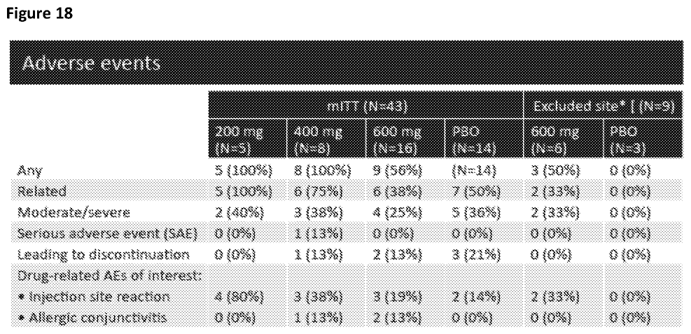

Rates of moderate-to-severe Adverse events (AEs) were comparable between 600 mg and placebo. AEs related to treatment were similar between groups (FIG. 18).

Interestingly, AEs leading to treatment discontinuation were higher in the placebo group. 1 serious adverse event (SAE) reported in the study (mild abdominal pain, 400 mg); considered unrelated to treatment No deaths were reported.

### CONCLUSION

This study demonstrates that eblasakimab was well tolerated with significant improvements vs. placebo in several efficacy outcomes in a Phase 1b study in adults with moderate to severe AD. In particular, a significant improvement in sleep disturbance score was observed in patients treated with eblasakimab vs patients treated with placebo. Robustness of the data from the small study was supported by sensitivity analyses on the primary analysis set. Including the Excluded site data did not change the primary endpoint or conclusions.

That these significant improvements were seen within the 8-week study period offers the potential for a greater magnitude of effect with prolonged treatment, supporting further investigation in an ongoing Phase 2b clinical trial.

### Example 4

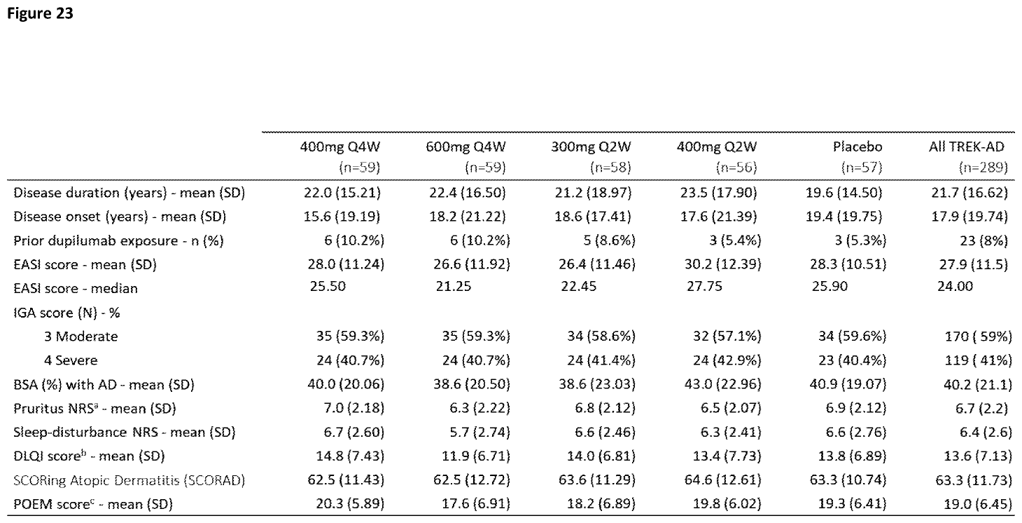

The TREK-AD (TRials with EblasaKimab in Atopic Dermatitis) study is a randomized, double-blind, placebo-controlled, dose-ranging Phase 2b clinical trial, designed to evaluate the efficacy and safety of eblasakimab as monotherapy in biologic-naïve adult patients with moderate-to-severe AD who are candidates for systemic therapy. The baseline characteristics of the Intention to Treat (ITT) group are shown in FIG. 23.

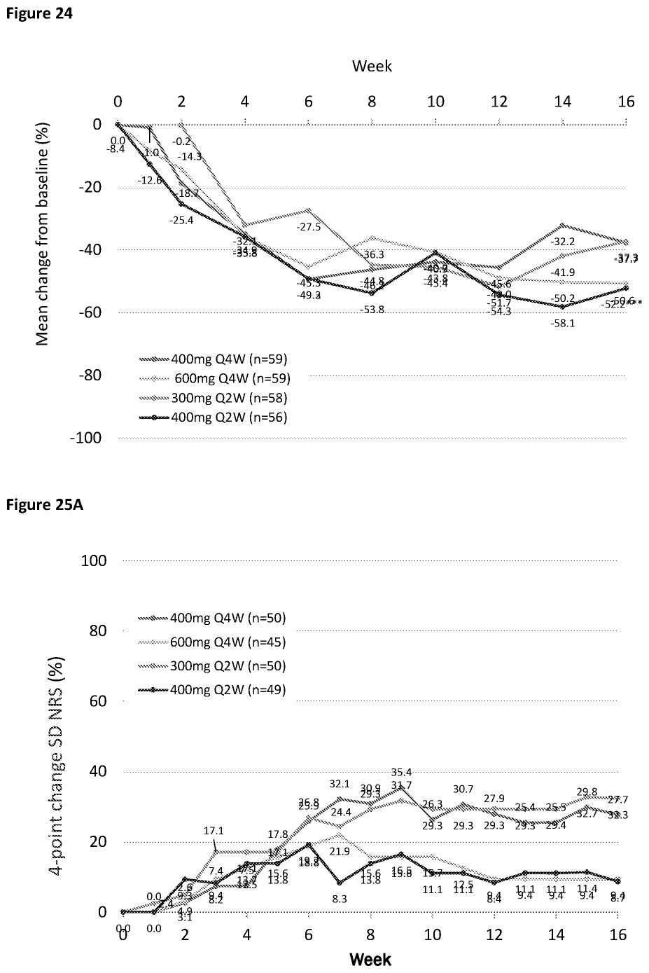

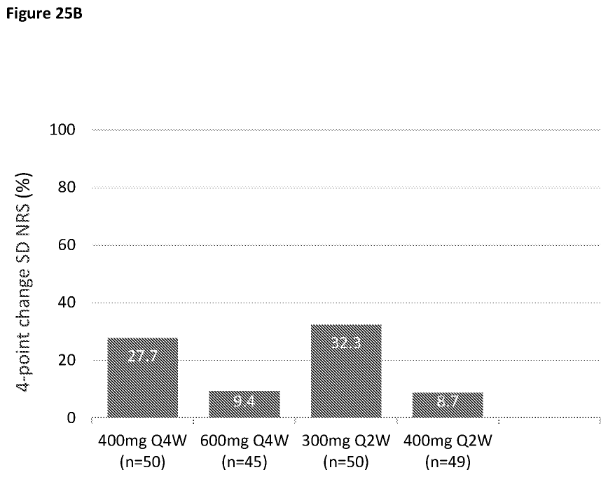

Various clinical analyses were performed during the TREK-AD study, including the change in EASI, SCORAD, DLQI, POME, EQ-5D-5L and HAD, and proportion of patients achieving a 4 point-reduction in SD-NRS from baseline to week 16. The results of the change in sleep deprivation component of SCORAD and proportion of patients achieving a 4 point-reduction in SD-NRS from baseline to week 16 are shown in FIGS. 24 and 25 respectively.

FIG. 24 clearly demonstrates that eblasakimab improves sleep disturbance as measured by the individual component scores of the SCORAD quality of life measure. In particular, eblasakimab at 400 mg Q4W, 600 mg Q4W, 300 mg Q2W, and 400 mg Q4W showed −37.7%, −50.6%, −37.3% and −52.2% change from baseline in sleep loss scores, respectively.

FIG. 25 shows that a significant proportion of patients were able to achieve a 4 point or more reduction (a 4 point reduction in SD-NRS represents a clinically significant improvement) in SD-NRS from baseline at week 16. In particular, eblasakimab at 300 mg Q2W showed 32.3%, i.e. almost a third of the patients in this treatment arm had a change of 4 points or more in SD-NRS.

Thus, the results of the TREK-AD study further demonstrate that eblasakimab has the potential to significantly improve sleep loss/sleep deprivation in patients.

1.-26. (canceled)

27. A method for treating sleep loss or sleep disturbance in a patient, comprising administering parentally a treatment cycle comprising a dose in the range 200 mg to 600 mg, (for example 300, 400, 500 or 600 mg, such as 400 to 600 mg) of an antibody or antigen binding fragment thereof, which is an inhibitor of signalling through IL-13Rα1 by binding the said receptor, for example wherein the treatment results in a reduction in the sleep disturbance (SD) score of the Patient Oriented Eczema Measure (POEM), for example in the range −50 to −100% from the baseline, such as a reduction of 2 to 4 points from the baseline.

28. The method according to claim 27, wherein the treatment results in a reduction in the sleep disturbance (SD) score of the Patient Oriented Eczema Measure (POEM), for example in the range −50 to −100% from the baseline, such as a reduction of 2 to 4 points from the baseline.

29. The method according to claim 27, wherein the patient has a baseline SD score of at least 3, such as 3 or 4.

30. The method according to claim 27, wherein the reduction in SD score is 2 to 4 points from the baseline, such as 2, 3 or 4.

31. The method according to claim 27, wherein the treatment results in a reduction in the sleep deprivation component of SCORAD, for example in the range of −20 to −100% from the baseline, such as a reduction of 2 or more points, such as 2, 3, 4, 5, 6, 7, 8, 9 or 10 points from the baseline.

32. The method according to claim 27, wherein
the treatment results in a reduction in the sleep deprivation component of SCORAD, for example in the range of −20 to −100% from the baseline, such as a reduction of 2 or more points, such as 2, 3, 4, 5, 6, 7, 8, 9 or 10 points from the baseline; and
the patient has a baseline sleep deprivation component of SCORAD of at least 4, such as 4, 5, 6, 7, 8, 9 or 10.

33. The method according to claim 27, wherein
the treatment results in a reduction in the sleep deprivation component of SCORAD, for example in the range of −20 to −100% from the baseline, such as a reduction of 2 or more points, such as 2, 3, 4, 5, 6, 7, 8, 9 or 10 points from the baseline; and/or
the patient has a baseline sleep deprivation component of SCORAD of at least 4, such as 4, 5, 6, 7, 8, 9 or 10; and
wherein the reduction in the sleep deprivation component of SCORAD is in the range of −30 to −60% from the baseline, such as −30%, −35%, −40%, −45%, −50%, −55% or −60%.

34. The method according to claim 27, wherein
the treatment results in a reduction in the sleep disturbance numerical rating scale (SD NRS), for example in the range of −20 to −100% from the baseline, such as a reduction of 4 or more points, such as 4, 5, 6, 7, 8, 9 or 10 points from the baseline; and/or
the patient has a baseline SD NRS of at least 4, such as 4, 5, 6, 7, 8, 9 or 10 points from the baseline.

35. The method according to claim 27, wherein
the treatment results in a reduction in the sleep deprivation component of SCORAD, for example in the range of −20 to −100% from the baseline, such as −30 to −60% from the baseline, such as −30%, −35%, −40%, −45%, −50%, −55% or −60%, such as a reduction of 2 or more points, such as 2, 3, 4, 5, 6, 7, 8, 9 or 10 points from the baseline; and/or
the patient has a baseline sleep deprivation component of SCORAD of at least 4, such as 4, 5, 6, 7, 8, 9 or 10; and
wherein there is a reduction of 4 or more points in SD NRS, as 4, 5, 6, 7, 8, 9 or 10 points from the baseline.

36. The method according to claim 27, wherein the patient is a highly allergic patient, for example where the baseline IgE levels have been established and are at a level of at least 10,000 KU/L+/−2,000.

37. The method according to claim 27, wherein the reduction in SD score, sleep deprivation score of SCORAD or SD NRS is observed by about day 15.

38. The method according to claim 27, wherein the patient has pruritus and/or a skin condition, for example selected from the group comprising dermatitis; such as atopic dermatitis (AD) (for example moderate to severe atopic dermatitis), contact dermatitis, neurodermatitis or seborrheic dermatitis; eczema; hand-foot-and-mouth disease; hives (including urticaria associated with Lupus), psoriasis, an infection, such as a fungal or bacterial infection, for example impetigo or folliculitis; an allergic skin reaction; Ehlers-Danlos syndrome; asthma; and angioedema, such as hereditary angioedema (HAE).

39. The method according to claim 27, wherein the treatment is administered subcutaneously.

40. The method according to claim 27, wherein multiple doses are administered in a treatment cycle (for example wherein the treatment cycle is 4 to 8 weeks, such as 8 weeks).

41. The method according to claim 27, wherein multiple treatment cycles are administered, for example 2, 3, 4 or more treatment cycles are administered.

42. The method according to claim 27, wherein said antibody or binding fragment thereof is administered either:
(i) once approximately every two weeks, (in particular a single treatment cycle, especially 8 weeks); or
(ii) administered once approximately every four weeks (for example monthly), (in particular a single treatment cycle, especially 8 weeks).

43. The method according to claim 27, wherein a loading dose in the range 400 to 900 mg, for example 400, 500, 600, 700, 800 or 900 mg is employed before administration of the treatment cycle.

44. An antibody or antigen binding fragment thereof for use according to claim 27, wherein the dose is in the range of from 300 mg to 600 mg, such as 300 mg, 400 mg, or 600 mg.

45. The method according to claim 27, wherein the antibody comprises a VL domain comprising an amino acid sequence shown in SEQ ID NO: 53 or a sequence at least 95% identical thereto, in particular SEQ ID NO: 53, and a VH comprising an amino acid sequence shown in SEQ ID NO: 51 or a sequence at least 95% identical thereto, in particular SEQ ID NO: 51.

46. The method according to claim 27, wherein the antibody is eblasakimab.
